(19) United States
(12) Patent Application Publication
(10) Pub. No.: US 2004/0023314 A1
(43) Pub. Date: Feb. 5, 2004

(54) Mutant fibronectin and tumor metastasis

(75) Inventor: Rong-Fu Wang, Houston, TX (US)

Correspondence Address: FULBRIGHT & JAWORSKI, LLP, 1301 MCKINNEY, SUITE 5100, HOUSTON, TX, 77010-3095, US

(73) Assignee: Baylor College of Medicine, Houston, TX

(21) Appl. No.: 10/447,161

(22) Filed: May 28, 2003

Related U.S. Application Data
(60) Provisional application No. 60/383,530, filed on May 28, 2002.

(51) Int. Cl. G01N 33/574

(52) U.S. Cl. 435/7.23

(57) ABSTRACT

The present invention relates to a mutated fibronectin as a class II-restricted tumor antigen recognized by tumor-reactive CD4+ T cells. In a specific embodiment, the mutation in fibronectin is responsible for the loss of FN matrix formation, leading to the enhanced migration of tumor cells. This provides an exemplary important immune target for effective cancer immunotherapy.

### CROSS-REFERENCE TO THE RELATED APPLICATIONS

This application claims priority to U.S. Provisional Patent Application 60/383,530, filed May 28, 2002, which is incorporated by reference herein in its entirety.

### STATEMENT REGARDING FEDERALLY SPONSORED RESEARCH OR DEVELOPMENT

The present invention utilized federal funds pursuant to NIH Grant No. R01CA90327. The United States Government may have certain rights in the invention.

### FIELD OF THE INVENTION

The present invention regards the fields of cell biology, molecular biology, and medicine.

### BACKGROUND OF THE INVENTION

Cancer cells are derived from a cell with accumulated genetic mutations or alterations, making them more immunogenic than normal cells. Although a number of tumor antigens recognized by CD8+ T cells have been identified in melanomas as well as other types of cancers, the majority of these class I-restricted antigens are nomnutated self-proteins (Boon et al., 1994; Wang and Rosenberg, 1999; Houghton et al., 2001). Few mutated antigens, including CDK4, beta-catenin and caspase 8, have been identified and implicated in the involvement of cell cycle regulation, tumorigenesis or apoptosis (Wolfel et al., 1995; Robbins et al,. 1996). To facilitate the identification of MHC class II-restricted antigens, the present inventor recently developed a novel genetic approach to cloning the genes encoding MHC class II-restricted tumor antigens (Wang et al., 1999). Three class II-restricted tumor antigens were successfully identified by this method: fusion protein LDFP resulting from chromosomal rearrangement, and the mutated antigens CDC27 and TPI, while the latter being independently identified by a biochemical approach (Wang et al., 1999; Wang et al., 1999; Pieper et al., 1999). Of particular interest, the mutated human CDC27 protein, an important component of an anaphase promoting complex involved in cell cycle regulation, could give rise to a melanoma target antigen, although the point mutation itself does not constitute a T-cell epitope. Instead, the missense mutation in a putative phosphorylation site allows a nonmutated peptide within CDC27 to be presented to T cells by MHC class II molecules (Wang et al., 1999). Indeed, the majority of MHC class II-restricted tumor antigens identified to date with use of tumor reactive T cells are mutated or fusion proteins, and therefore may represent immunogenic targets recognized by CD4+ T cells.

The biological functions of the mutated antigens identified by tumor reactive CD4+ and CD8+ T cells suggest that these mutated gene products contribute not only to tumor development, but also to tumor metastasis and progression. Although melanoma is a highly invasive skin cancer, few genetic mutations have been linked to its biological behavior. It is widely accepted that after the onset of oncogenesis, tumor cells with metastatic potential migrate away from the primary tumor, invade and implant in distant sites, where they re-establish tumor growth (Clark et al., 2000). Gene products involved in the formation of extracellular matrix (ECM), including fibronectin (FN), have been implicated in tumorigenesis and metastasis (Hynes et al., 1978; Giancotti and Ruoslahti, 1990; Akamatsu et al., 1996), but so far no direct evidence for this association has been reported (Clark et al., 2000).

Metastasis accounts for most deaths in cancer patients with solid tumors. The extent of tumor cell adhesion to the extracellular matrix and the stimulation of angiogenesis are critical steps in the metastatic process. Metastatic potential depends upon a complex series of events, including interactions of malignant tumor cells with the extracellular matrix. Cellular adhesion to the extracellular matrix is primarily mediated by integrins, which are cell surface receptors that comprise an expanding family of transmembrane heterodimers of an α and β subunit. The identity of the subunits usually determines the receptor's functional specificity.

One of the most important integrin ligands and a major component of extracellular matrix is fibronectin. Superfibronectin (sFN) is a polymeric fibrillar form of fibronectin that may be related to the natural matrix form of fibronectin. Compared to fibronectin, sFN has greatly enhanced cell adhesive properties. Whereas cells attach to fibronectin through integrins, cell attachment to sFN is mediated by both integrins and other distinct receptors. sFN can be produced in vitro by the incubation of fibronectin with fibronectin fragments. Morla, A., et al., Nature 367:193-196 (1994).

In U.S. Pat. No. 6,475,488, there is a method of inhibiting cancer in a subject by administering a superfibronectin-generating compound to the subject, wherein the superfibronectin-generating compound comprises a III₁-C or III-1C polypeptide fragment of fibronectin consisting of a particular sequence. However, this patent does not address the need for a class II-restricted tumor antigen recognized by CD4+ cells, and particularly one in which a marker is provided for a tumor cell that will metastasize. Thus, the present invention addresses a long-felt need in the art to provide a class II-restricted tumor antigen recognized by CD4+ cells, particularly one comprising mutant fibronectin, that is related to the diagnosis and treatment of cancer, such as metastasis of cancer.

### BRIEF SUMMARY OF THE INVENTION

The present invention is directed to a system and method that relates to the identification and characterization of mutant FN as a class II-restricted tumor antigen recognized by tumor-reactive CD4+ T cells derived from a patient having cancer. In a specific embodiment, the cancer is melanoma. As described in the Examples herein, an exemplary mutation in FN gave rise to an epitope for T-cell recognition. Although any mutation of fibronectin is within the scope of the invention, in one specific embodiment, the mutation is a point mutation that results in the substitution of a Lys (positively charged) for Glu (negatively charged) residue in FN, giving rise to the epitope for T-cell recognition. Importantly, the mutation in FN was directly responsible for the loss of FN matrix formation, leading to the enhanced migration of tumor cells. Thus, identification of such MHC class II-restricted tumor antigens not only provides potentially important immune targets for effective cancer immunotherapy, but also improves understanding of the mechanisms by which these antigens participate in tumor development and metastasis.

In one embodiment of the present invention, there is an isolated mutated fibronectin that acts as a tumor antigen recognized by DC4+ T cells, and it plays a role in extracellular matrix formation and/or tumor metastasis.

In one embodiment of the present invention, there is a method of identifying a cell that will differentiate into a metastatic cancer cell, comprising the step of identifying a mutated fibronectin in said cell, such as, for example, a cell in a tumor. The mutated fibronectin may be a mutated fibronectin polynucleotide, such as, for example, one that comprises a G to A mutation at position 6427. Thus, in some embodiments of the present invention the mutated fibronectin is a diagnostic marker of a cancer cell, such as a metastatic cancer cell.

In other embodiments, the mutated fibronectin is a mutated fibronectin polypeptide, which may comprise a Glu to Lys substitution at position 2053. It may also further be defined as comprising the sequence MIFEKHGFRRTTPP (SEQ ID NO: 1). In a specific embodiment, the metastatic tumor cell is a melanoma cell, a prostate cancer cell, a breast cancer cell, a lung cancer cell, an ovarian cancer cell, a brain cancer cell, a liver cancer cell, a colon cancer cell, or a kidney cancer cell. In a further specific embodiment, the metastatic tumor cell is a melanoma cell.

In another embodiment of the present invention, there is a method of predicting metastasis from a cancer, comprising the step of identifying a mutated fibronectin polynucleotide or polypeptide in at least one cell of the cancer. In a specific embodiment, the mutated fibronectin polynucleotide comprises a G to A mutation at position 6427. In a further specific embodiment, the mutated fibronectin polypeptide comprises a Glu to Lys substitution at position 2053 and/or may be defined as comprising the sequence MIFEKHGFRRTTPP (SEQ ID NO: 1). In a specific embodiment, the cancer is melanoma.

In an additional embodiment of the present invention, there is a method of eliciting an immune response in an individual, comprising the step of delivering to the individual a vector comprising a mutant fibronectin. In a specific embodiment, the mutant fibronectin is further defined as a mutant fibronectin polynucleotide, such as, for example, one that comprises a G to A mutation at position 6427. Alternatively, the mutant fibronectin is further defined as a mutant fibronectin polypeptide, such as one that comprises a Glu to Lys substitution at position 2053 and/or the sequence MIFEKHGFRRTTPP (SEQ ID NO: 1).

The mutant fibronectin may be delivered to the individual in a cell, such as an immune effector cell, such as a dendritic cell. Furthermore, in some embodiments following the delivery of the mutant fibronectin to the immune effector cell at least part of a mutant fibronectin polypeptide is presented on the cell surface. The method may further comprise the step of delivering a tumor antigen other than mutant fibronectin to the individual, such as by being delivered in a cell. In some embodiments, the tumor antigen is TRP-2 or NY-ESO-1.

In an additional embodiment of the present invention, there is a method of preventing metastasis of a cancer in an individual, wherein the individual comprises at least one cell having a mutated fibronectin, comprising the step of stimulating an immune response against at least said cell. In a specific embodiment, the immune response is further defined as being against said mutated fibronectin. In another specific embodiment, the cell having a mutated fibronectin is a cancer cell. In one embodiment, the stimulating an immune response is further defined as comprising the steps of introducing the mutated fibronectin to a dendritic cell; and administering the cell comprising the mutated fibronectin to the individual, wherein the dendritic cell presents at least part of the mutated fibronectin on its surface.

In further embodiments, there is an isolated mutant fibronectin polynucleotide comprising a G to A mutation at position 6427, an isolated mutant fibronectin polypeptide comprising a Glu to Lys substitution at position 2053, an isolated mutant fibronectin polypeptide comprising MIFEKHGFRRTTPP (SEQ ID NO: 1), and/or a pharmaceutical composition comprising a mutant fibronectin polynucleotide having a G to A mutation at position 6427. In a specific embodiment, the polynucleotide is in a pharmaceutically acceptable carrier.

In additional embodiments, there is a pharmaceutical composition comprising a mutant fibronectin polypeptide having a Glu to Lys substitution at position 2053. In a specific embodiment, the polypeptide comprises MIFEKHGFRRTTPP (SEQ ID NO:1). In another specific embodiment, the polypeptide is in a pharmaceutically acceptable carrier.

In further embodiments, there is an immunological composition comprising a mutant fibronectin polynucleotide having a G to A mutation at position 6427 and/or an immunological composition comprising a mutant fibronectin polypeptide having a Glu to Lys substitution at position 2053, such as a polypeptide comprising MIFEKHGFRRTTPP (SEQ ID NO:1).

In additional embodiments, there is an immune effector cell comprising a mutant fibronectin polynucleotide having a G to A mutation at position 6427, and the cell may be a dendritic cell. In further embodiments, there is an immune effector cell comprising a mutant fibronectin polypeptide having a Glu to Lys substitution at position 2053, and the polypeptide may comprise MIFEKHGFRRTTPP (SEQ ID NO: 1). In a specific embodiment, the cell is a dendritic cell.

In further embodiments there is an antigen presenting cell transduced with a vector comprising a mutant fibronectin polynucleotide having a G to A mutation at position 6427 or an antigen presenting cell comprising a fibronectin polypeptide having the sequence MIFEKHGFRRTTPP (SEQ ID NO: 1).

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIGS. 1A and 1B illustrate specific recognition of autologous melanoma cells by CD4+ F27TIL-T1. In FIG. 1A, there is specific antitumor recognition of CD4+ F27TIL-T1. F27TIL-T1 recognized the autologous F27 mel cells, but not EBV-B cell lines and allogeneic melanoma cell lines or 293-derived cell lines. 888 mel, 1011 mel, 1195 mel, 1280 mel, 1297 mel and 1390 mel shared the DR2 molecule with F27 mel. T cell recognition was evaluated by GM-CSF release from CD4+ F27TIL-T1. FIG. 1B provides HLA restriction of T cell recognition. CD4+ F27TIL-T1 cells were co-cultured with autologous F27 mel cells in the presence or absence of various anti-MHC antibodies. GM-CSF release was determined after an 18 h incubation. T cell recognition of F27 mel was specifically blocked by an anti-DR antibody, but not by anti-MHC class I, anti-DQ or anti-DP antibodies.

FIGS. 2A and 2B show screening of an Ii-cDNA library from RNA of F27 mel using CD4+ F27TIL-T1 cells. In FIG. 2A, there is identification of positive cDNA clones encoding a tumor antigen recognized by CD4+ T cells. After screening of 2×10 Ii-cDNA fusion library clones generated from F27 mel RNA, positive cDNA pools were identified on the basis of GM-CSF release from CD4+ F27TIL-T1. CD4+ F27TIL-T1 recognized 293IMDR2 transfected with the cDNA clone 3, but not with a control cDNA or GFP. FIG. 2B shows an exemplary schematic presentation of FN protein and the position of the mutated amino acid.

FIGS. 3A and 3B demonstrate identification and characterization of peptides capable of stimulating CD4+ T cells. FIG. 3A shows identification of peptides recognized by F27TIL-T1 cells. Two of three overlapping peptides that contain the mutated amino acid residue were recognized by T cells, while the corresponding peptide with a wild-type residue failed to stimulate CD4+ T cells. FIG. 3B shows determination of peptide concentrations required for T cell recognition. 293IMDR2 cells were incubated with different concentrations of FN-P2 peptide for 90 min, and then washed 3 times with T cell assay medium. T cells were added to peptide-pulsed 293IMDR2 cells overnight. GM-CSF release from T cells was determined with GM-CSF ELISA kit.

FIGS. 4A through 4C show loss of FN matrix formation in F27 mel cells harboring a mutated FN. In FIG. 4A there is immunostaining of FN matrix of three melanoma cell lines 1143 mel, 1195 mel and F27 mel with Ab-9 anti-FN antibody. FN was detected by indirect immunofluorescence. Phase contrast and fluorescence images were taken at 40× magnification. FIG. 4B shows genomic DNA sequence analysis of FN in three melanoma cell lines. Genomic DNA fragments were amplified by FN-specific primers. The PCR products were sequenced to identify mutated FN. FIG. 4C provides comparison of immunostaining for FN matrix in 1143 mel and F27 mel cells with different anti-FN (Ab-1, Ab-7, AB-8 and Ab-9) antibodies that recognize epitopes in the different regions of FN. DAPI staining was used as controls for cell density. Staining of FN matrix in 1143 mel and F27 mel cells with all four antibodies showed similar patterns: intensive staining of FN in 1143 mel cells, but little or weak staining in F27 mel cells.

FIG. 5 illustrates the migratory capacity of F27 mel compared with 1143 mel and 1195 mel cells. The poorly metastatic tumor cell lines 1143 mel and 1195 mel have little or no capacity to migrate from the seeded chamber to the chamber with complete medium, while F27 mel readily migrated to the chamber with complete medium. Cells were stained with crystal violet, examined under a compound microscope and photographed.

FIGS. 6A though 6C provide western and Northern blot analyses of FN in different tumor cell lines. FIG. 6A shows that tumor cell lysates of 1143 mel and F27 mel were first immunoprecipitated with an anti-beta 1 integrin. The immunoprecipitated proteins were separated on a SDS-PAGE. After transfer to membrane, proteins were detected with anti-FN or anti-beta integrin antibodies. In FIG. 6B, whole tumor cell lysates of 1143 mel and F27 mel cells were separated by SDS-PAGE and analyzed with the anti-FN antibody to determine the total FN protein in the tumor cell lysates. FIG. 6C shows Northern blot analysis of total RNA isolated from different tumor cell lines 1143 mel and F27 mel cells. Hybridization of blots with the probe of FN detected an 8 kb band in 1143 mel and F27 mel tumor cells. An actin probe was used to verify that equal amounts of total RNA were loaded for each well. FN-specific RNA in F27 mel cells was at least 3-to-4 fold higher than that in 1143 mel cells.

FIGS. 7A through 7B provide determination of the FN mutation responsible for the dominant-negative phenotype of FN matrix formation. In FIG. 7A, PCR products (7114 bp) of full-length FN were obtained after amplification using a pair of primers and the first strand cDNA generated from F27 mel tumor RNA. Cloning of both wild-type and mutant FN in a pcDNA3 expression vector. Wild-type and mutant FN were verified by DNA sequencing. FIG. 7B shows immunostaining of FN matrices in 1143 mel and derivative cell lines expressing wild-type, mutant FN gene, or empty vector. DAPI staining was used a control for cell density.

FIGS. 8A through 8B show that mutant FN is responsible for the enhanced metastatic potential of tumor cells. FIG. 8A shows that stable expression of mutant FN in 1143 mel cells resulted in enhanced migration ability in matrigel assays. Untreated 1143 mel and 1143 mel cells expressing wild-type FN or empty vector showed little or no migration from one chamber to another containing complete medium. In FIG. 8B, migration assays were repeated in three independent experiments, each comprising four sets of independent 1143 mel-derived tumor cell clones expressing wild-type, mutant FN or empty vector. The numbers of cells that migrated to the bottomed chamber containing complete medium were counted in four representative fields (20×) per chamber. The data are presented as means of number of cells migrated and standard deviations. A P-value of 0.0007 was obtained from a t-Test for the group expressing mutant FN and any other groups.

### DETAILED DESCRIPTION OF THE INVENTION

The practice of the present invention will employ, unless otherwise indicated, conventional techniques of molecular biology, microbiology, recombinant DNA, and immunology, which are within the skill of the art. Such techniques are explained fully in the literature. See e.g., the following references and particularly their updated versions: Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), ANIMAL CELL CULTURE (R. I. Freshney, Ed., 1987), the series METHODS IN ENZYMOLOGY (Academic Press, Inc.); GENE TRANSFER VECTORS FOR MAMMALIAN CELLS (J. M. Miller and M. P. Calos eds. 1987), HANDBOOK OF EXPERIMENTAL IMMUNOLOGY, (D. M. Weir and C. C. Blackwell, Eds.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987), CURRENT PROTOCOLS IN IMMUNOLOGY (J. E. Coligan, A. M. Kruisbeek, D. H. Margulies, E. M. Shevach and W. Strober, eds., 1991); ANNUAL REVIEW OF IMMUNOLOGY; as well as monographs in journals such as ADVANCES IN IMMUNOLOGY. All patents, patent applications, and publications mentioned herein, both supra and infra, are hereby incorporated herein by reference.

U.S. Patent Application Ser. No. 10/077,555, filed Feb. 15, 2002, is incorporated by reference herein in its entirety.

Definitions

As used herein the specification, “a” or “an” may mean one or more. As used herein in the claim(s), when used in conjunction with the word “comprising”, the words “a” or “an” may mean one or more than one. As used herein “another” may mean at least a second or more.

The term “dendritic cell” refers to any member of a diverse population of morphologically similar cell types found in lymphoid or non-lymphoid tissues. These cells are characterized by their distinctive morphology, high levels of surface MHC-class II expression (Steinman et al., 1991). These cells can be isolated from a number of tissue sources, as is well known in the art. Furthermore, a skilled artisan recognizes that antigen-pulsed dendritic cells have traditionally been prepared in one of two exemplary ways: (1) small peptide fragments, known as antigenic peptides, are “pulsed” directly onto the outside of the APCs (Mehta-Damani et al., 1994); or (2) APCs are incubated with whole proteins or protein particles which are then ingested by the APCs. These proteins are digested into small peptide fragments by the APC and eventually carried to and presented on the APC surface (Cohen et al., 1994).

The cell surface of dendritic cells is unusual, with characteristic veil-like projections, and is characterized by having the cell surface markers CD1+, CD4+, CD86+, or HLA-DR+. Dendritic cells have a high capacity for sensitizing MHC-restricted T cells and are very effective at presenting antigens to T cells in situ, both self-antigens during T cell development and tolerance and foreign antigens during immunity.

Because of their effectiveness at antigen presentation, there is growing interest in using dendritic cells ex vivo as tumor or infectious disease vaccine adjuvants (see, for example, Romani et aL., (1994)). The use of dendritic cells as immunostimulatory agents has been limited due to the low frequency of dendritic cells in peripheral blood, the limited accessibility of lymphoid organs and the dendritic cells' terminal state of differentiation. Dendritic cells originate from CD34+ bone marrow or peripheral blood progenitors and peripheral blood mononuclear cells, and the proliferation and maturation of dendritic cells can be enhanced by the cytokines GM-CSF (sargramostim, Leukine®, Immunex Corporation, Seattle, Wash.), TNF-α, c-kit ligand (also known as stem cell factor (SCF), steel factor (SF), or mast cell growth factor (MGF)) and interleukin-4. Flt3-L has been found to stimulate the generation of large numbers of functionally mature dendritic cells, both in vivo and in vitro (U.S. Ser. No. 08/539,142, filed Oct. 4, 1995).

The term “immune effector cell” as used herein is defined as any cell that is capable of eliciting a T cell response in an animal and that is capable of uptake, and preferably also presentation, of an antigen, wherein the antigen is delivered to the cell via a cell penetrating peptide.

The term “immunological composition” as used herein is defined as a composition capable at least in part of eliciting any kind of immune response in an individual. In a specific embodiment, the immunological composition is a vaccine.

The term “mature dendritic cell” as used herein is defined as dendritic cells that express a high level of MHC class II, CD80 (B7.1) and CD86 (B7.2) molecules, while immature dendritic cells express low levels of MHC class II, CD80 (B7.1) and CD86 (B7.2) molecules but have a great capacity of uptaking antigens.

As used herein, the terms “metastasis” and “metastases” refer to the movement of a tumor cell from its primary site by any means or by any route, including local invasion, lymphatic spread, vascular spread or transcoelomic spread.

The term “therapeutically effective” as used herein refers to the amount of a compound required to improve some symptom associated with a disease. For example, in the treatment of cancer, a compound that improves the cancer to any degree or arrests any symptom of the cancer would be therapeutically effective. For example, the improvement of the cancer may be inhibition of metastasis of a cancer (such as a tumor), inhibition of angiogenesis of a cancer cell and/or tissue, inhibition or retardation of cell growth, facilitation of cell death, or a combination thereof. The inhibition need not be complete, although in some embodiments it may be. A therapeutically effective amount of a compound is not required to cure a disease but will provide a treatment for a disease.

The terms “tumor antigen, or tumor rejection antigen, or tumor associated antigen” as used herein refer to an antigen capable of eliciting an immune response in an animal to a tumor. In another specific embodiment, the antigen is on the surface of malignant cells, is unique to the cancerous cells and is not present on their normal counterparts. In a specific embodiment, the anti-tumor antigen is a tumor-associated antigen, which is an antigen present on both normal and cancerous cells but ‘hidden’ on normal cells, becoming ‘visible’ when malignant, or overexpressed on the latter, as a product of cellular oncogenes.

The Present Invention

CD4+ T cells play an important role in orchestrating host immune responses against cancer, particularly in providing critical help for priming and extending the survival of CD8+ T cells. However, relatively little is known about MHC class II-restricted human tumor antigens capable of activating CD4+ T cells. Here, the identification of a mutated fibronectin (FN) as a tumor antigen recognized by HLA-DR2-restricted CD4+ T cells is described. DNA sequencing analysis indicated that this gene contains a mutation, resulting in the substitution of lysine for glutamic acid and giving rise to a new T-cell epitope recognized by CD4+ T cells. Tumor cells harboring the mutant FN resulted in the loss of FN matrix formation, and gained metastatic potential based on the migration pattern compared with that of tumor cells expressing wild-type FN. Further studies using cell lines stably expressing the mutated FN cDNA demonstrated that the point mutation in FN was responsible for the loss of FN staining in extracellular matrices and the enhancement of tumor cell migration. These findings represent the first demonstration that a mutated gene product recognized by CD4+ T cells is directly involved in tumor metastasis, showing the importance of CD4+ T cells in controlling the spread of tumor cells to distant anatomic sites.

The present invention utilizes a mutant fibronectin as a tumor antigen recognized by CD4+ T cells and, in some embodiments, it has a role in extracellular matrix formation and tumor metastasis. A skilled artisan recognizes the fibronectin may be mutated in any position, although in a preferred embodiment it is mutated at position 6427. In a specific embodiment, it is a G to A mutation at position 6427. In some embodiments, there may be multiple mutations. The mutation can be a point mutation, a deletion, an inversion, a frame shift mutation, or a combination thereof, and others well known in the art. The mutation may be in a protein-binding domain. An immune response may be elicited upon a naturally occurring mutant fibronectin or through an exogenously derived mutant fibronectin, such as generated by the hand of man.

In some embodiments of the present invention, the function of the mutated fibronectin is altered. In other embodiments, the protein stability of the polypeptide is altered directly or indirectly because of the mutation. Some exemplary functions that may be altered include its association with the extracellular matrix and/or its association with a complex of polypeptides, such as specific protein-binding being altered. For example, the protein stability of the mutant fibronectin may be affected, which affects its ability to complex with other proteins, such as, for example, fibronectin (wild-type), integrins, heparin, low density lipoprotein receptor-related protein (LRP) and/or matrix metalloproteinase-2 (MMP-2).

In some embodiments, a fibronectin nucleic acid or polypeptide sequence is mutated for utilization in the present invention. Exemplary fibronectin polynucleotides in which the mutation may be generated include (followed by their GenBank Accession Number): SEQ ID NO:2 (M10905); SEQ ID NO:4 (E01162); and SEQ ID NO:8 (NM₁₃ 002026).

An exemplary fibronectin polypeptide includes (followed by its GenBank Accession Number): SEQ ID NO:3 (NP_{—}002017).

As indicated, the present invention utilizes a mutant fibronectin as a tumor antigen recognized by CD4+ T cells. In some embodiments of the present invention, the mutant fibronectin is administered to an individual with cancer, which may be any form of cancer. In specific embodiments, the cancer is metastatic cancer. In other specific embodiments, the cancer is melanoma.

The mutant fibronectin may be administered by any appropriate means known in the art. In one specific embodiment, it is administered comprised on or with a vector and may be nucleic acid or polypeptide in form. The nucleic acid or polypeptide may be administered in a pharmaceutically acceptable carrier. In some embodiments, it is administered in a cell, such as an immune effector cell, preferably with the mutant fibronectin epitope presented to the outside of the immune effector cell. In a particular aspect of the present invention, the nucleic acid or polypeptide comprising the mutant fibronectin is administered in a dendritic cell.

The mutant fibronectin may also be presented to an individual as a vaccine, such as a peptide vaccine, and preferably with an appropriate adjuvant, that is well known in the art, such as Freund's adjuvant. The individual need not have cancer to be administered the vaccine, although in some embodiments the individual does have cancer, and in further specific embodiments the cancer is metastatic.

The mutant fibronectin, regardless of the form in which it is administered to an individual, may be administered to an individual in combination with other tumor-specific antigens or any other form of appropriate cancer therapy, which is well known to those in the art.

Tumor Antigens

In the context of the present invention, “tumor antigen” refers to antigens that are common to specific tumor types. In a specific embodiment, the tumor antigen of the present invention is a class II-restricted tumor antigen recognized by tumor-reactive CD4+ T cells.

The tumor antigen of the present invention may form part of, or may be derived from, cancers including but not limited to primary or metastatic melanoma, thymoma, lymphoma, sarcoma, lung cancer, liver cancer, non-Hodgkin's lymphoma, Hodgkins lymphoma, leukemias, uterine cancer, cervical cancer, bladder cancer, kidney cancer and adenocarcinomas such as breast cancer, prostate cancer, ovarian cancer, pancreatic cancer, and the like. In one embodiment, the tumor antigen of the present invention comprises one or more antigenic cancer epitopes immunologically recognized by tumor infiltrating lymphocytes (TIL) derived from a cancer tumor of a mammal.

Malignant tumors express a number of proteins that can serve as target antigens for an immune attack. These molecules include but are not limited to tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma, the tumor-specific idiotype immunoglobulin constitutes a truly tumor-specific immunoglobulin antigen that is unique to the individual tumor. B-cell differentiation antigens such as CD19, CD20 and CD37 are other candidates for target antigens in B-cell lymphoma. Some of these antigens (CEA, HER-2, CD 19, CD20, idiotype) have been used as targets for passive immunotherapy with monoclonal antibodies with limited success.

Thus, examples of tissue-specific tumor antigens include, but are not limited to prostatic acid phosphatase (PAP; associated with prostatic tumors), Melan-A/MART-1 (associated with melanoma; Coulie et al., 1994, J. Exp. Med. 180:35, Hawakami et al., 1994, PNAS 91:3515, Bakker et al., 1994, J. Exp. Med. 179:1005), tyrosinase/albino (associated with melanoma; Kawakami et al., 1994, J. Exp. Med.), and CD19, CD20 and CD37 (associated with lymphoma).

Likewise, oncogene product peptide antigens have been identified that are common to specific tumor types. These polypeptides will find use in the polypeptide complexes of the present invention as reagents that can be used generally to stimulate T-cell responses effective to react with tumors bearing such antigens. Oncogene product peptide antigens include but are not limited to HER-2/neu (Beckmann et al., 1992, Eur. J. Cancer 28:322) associated with human breast and gynecological cancers and carcinoembryonic antigen (CEA) associated with cancer of the pancreas.

The tumor antigen and the antigenic cancer epitopes thereof may be purified and isolated from natural sources such as from primary clinical isolates, cell lines and the like. The cancer peptides and their antigenic epitopes may also be obtained by chemical synthesis or by recombinant DNA techniques known in the arts. Techniques for chemical synthesis are described in Steward et al. (1969); Bodansky et al. (1976); Meienhofer (1983); and Schroder et al. (1965). In some embodiments, site-directed mutagenesis imparts a mutation on a polynucleotide that encodes the tumor antigen.

Furthermore, as described in Renkvist et al. (2001), there are numerous antigens known in the art. The following tables describe T cell-defined epitopes encoded by tumor antigens, and only those tumor antigens recognized by T cells (either cytotoxic CD8+ or helper CD4+) are listed. Although analogs or artificially modified epitopes are not listed, a skilled artisan recognizes how to obtain or generate them by standard means in the art. Other antigens, identified by antibodies and as detected by the Serex technology (see Sahin et al. (1997) and Chen et al. (2000)), are identified in the database of the Ludwig Institute for Cancer Research, which a skilled artisan can easily find on the World Wide Web.

In a specific embodiment, the tumor antigen is TRP2 peptide (SVYDFFVWL; SEQ ID NO:7).

The following table is from Renkvist et al. (2001).

TABLE 1

Class I HLA-restricted cancer/testis antigens. All of these
antigens were found to be expressed by normal spermatocytes and/or spermatogonia of
testis. Occasionally MAGE-3, MAGE-4 and the GAGE genes were found to be
expressed also in placenta (De Backer et al., 1999; Cox et al., 1994).
The NY-ESO-1 antigen was found to be expressed in normal ovary cells
(Chen et al., 1997).

| | | | | |
|---|---|---|---|---|
| | HLA | | | Tissue distribution |
| Gene | allele | Peptide epitope | Authors | among tumors^{a} |

| | | | | | |
|---|---|---|---|---|---|
| MAGE-A1 | A1 | EADPTGHSY | (SEQ ID NO:9) | Traversari et al. | Melanoma, breast |
| | | | 1992 | carcinoma, SCLC (De | |
| | | | | Plaen et al., 1999); De | |
| MAGE-A1 | A3 | SLFRAVITK | (SEQ ID NO:10 | Chaux et | Smet et al., 1994, van |
| | | | al. 1999a | der Bruggen et al., | |
| | | | | 1994a)—sarcoma, | |
| MAGE-A1 | A24 | NYKHCFPEI | (SEQ ID NO:11) | Fujie et al. 1999 | NSCLC (De Plaen et |
| | | | | al., 1999), De Smet et | |
| | | | | al., 1994)—thyroid | |
| MAGE-A1 | A28 | EVYDGREHSA | (SEQ ID NO:12) | Chaux et | medullary carconoma |
| | | | al. 1999a | (van der Bruggen et al., | |
| | | | | 1994a)—colon | |
| | | | | carcinoma (De Plaen et | |
| | | | | al., 1999)—laryngeal | |
| | | | | tumors (De Smet et al., | |
| | | | | 1994) | |
| MAGE-A1 | B37 | REPVTKAEML | (SEQ ID NO:13) | Tanzarella et al. | Melanoma, colon and |
| -A2, -A3, | | | 1999 | breast carcinomas, | |
| -A6 | | | | SCLC (De Plaen et al., | |
| | | | | 1999, De Smet et al., | |
| | | | | 1994, van der Bruggen | |
| | | | | et al., 1994a)—sarcoma, | |
| | | | | NSCLC (De | |
| | | | | Plaen et al., 1999, De | |
| | | | | Smet et al., 1994—thyroid | |
| | | | | medullary | |
| | | | | carcinoma, H/N tumors, | |
| | | | | bronchial SCC (van der | |
| | | | | Bruggen et al., 1994a)—laryngeal | |
| | | | | tumors (De | |
| | | | | Smet et al., 1994)—leukemias | |
| | | | | (De Plaen et | |
| | | | | al., 1994) | |
| MAGE-A1 | B53 | DPARYEFLW | (SEQ ID NO:14) | Chaux et | Melanoma, breast |
| | | | al. 1999a | carcinoma, | |
| | | | | SCLC (De Plaen et al., | |
| MAGE-A1 | Cw2 | SAFPTTINF | (SEQ ID NO:15) | Chaux et | 1999, De Smet et al., |
| | | | al. 1999a | 1994, Van den Eynde et | |
| | | | | al., 1999)—sarcoma, | |
| MAGE-A1 | Cw3 | SAYGEPRKL | (SEQ ID NO:16) | Chaux et | colon carcinoma, |
| | | | al. 1999a | NSCLC (De Plaen et | |
| | | | | al., 1999, De Smet et | |
| MAGE-A1 | Cw16 | SAYGEPRKL | (SEQ ID NO:17) | van der Bruggen et | al., 1994)—thyroid |
| | | | al. 1994b | medullary carcinoma | |
| | | | | (van der Bruggen et al., | |
| | | | | 1994a) | |
| MAGE-A2 | A2 | KMVELVHFL | (SEQ ID NO:18) | Visseren et | Melanoma, colon and |
| | | | al. 1997 | breast carcinomas, | |
| | | | | SCLC (De Plaen et al., | |
| MAGE-A2 | A2 | YLQVFGIEV | (SEQ ID NO:19) | Visseren et | 1999, De Smet et al., |
| | | | al. 1997 | 1994, van der Bruggen | |
| | | | | et al., 1994a)—sarcoma, | |
| MAGE-A2 | A24 | EYLQLVFGI | (SEQ ID NO:20) | Tahara et al. 1999 | sarcoma, NSCLC (De |
| | | | | Plaen et al., 1999, De | |
| | | | | Smet et al., 1994)—thyroid | |
| | | | | medullary | |
| | | | | carcinoma (van der | |
| | | | | Bruggen et al., 1994a)—laryngeal | |
| | | | | tumors | |
| | | | | (Lurquin et al., 1997)—leukemias | |
| | | | | (De Plaen et | |
| | | | | al., 1999) | |
| MAGE-A3 | A1 | EADIPIGHLY | (SEQ ID NO:21) | Gaugler et | Melanoma, colon and |
| | | | al. 1994 | breast carcinomas (De | |
| | | | | Plaen et al., 1999, van | |
| MAGE-A3 | A2 | FLWGPRALV | (SEQ ID NO:22) | van der Bruggen et | der Bruggen et al., |
| | | | al. 1994a | 1994a)—H/N tumors | |
| | | | | (Chen et al., 1997)—bronchial | |
| MAGE-A3 | A24 | TFPDLESEF | (SEQ ID NO:23) | Oiso et al. 1999 [ | bronchial SCC, thyroid |
| | | | | medullary and bladder | |
| | | | | carcinoma, sarcomas, | |
| MAGE-A3 | A24 | IMPKAGLLI | (SEQ ID NO:24) | Tanaka et al. 1997 | SCLC, NSCLC, (van |
| | | | | der Bruggen et al., | |
| | | | | 1994a)—leukemias (De | |
| MAGE-A3 | B44 | MEVDPIGHLY | (SEQ ID NO:25) | Hermann et | Smet et al., 1994) |
| | | | al. 1996 | | |
| | | | Fleischhauer et | | |
| | | | al. 1996 | | |
| MAGE-A3 | B52 | WQYFFPVIF | (SEQ ID NO:26) | Russo et al. 2000 | |
| MAGE-A4 | A2 | GVYDGREHTV | (SEQ ID NO:27) | Duffour et | Melanoma, NSCLC, |
| | | | al. 1999 | sarcomas, esophageal, | |
| | | | | colon and breast | |
| | | | | carcinomas (De Plaen | |
| | | | | et al., 1999) | |
| MAGE-A6 | A34 | MVKISGGPR | (SEQ ID NO:28) | Zorn and Hercent, | Melanoma, NSCLC, |
| | | | 1999b | colon carcinoma, | |
| | | | | leukemias (De Plaen et | |
| | | | | al., 1999) | |
| MAGE-A10 | A2 | GLYDGMEHL | (SEQ ID NO:29) | Huang et al. 1999 | Not defined |
| MAGE-A12 | Cw7 | VRIGHLYIL | (SEQ ID NO:30) | Panelli et al 2000 | Melanoma, myeloma, |
| | | | Heidecker et al. | brain tumors, sarcoma, | |
| | | | 2000 | leukemias, SCLC, | |
| | | | | NSCLC, H/N tumors, | |
| | | | | bladder, lung, | |
| | | | | esophageal, breast, | |
| | | | | prostate and colorectal | |
| | | | | carcinoma (De Plaen et | |
| | | | | al., 1994) | |
| BAGE | Cw16 | AARAVFLAL | (SEQ ID NO:31) | Boer et al. 1995 | Melanoma, bladder and |
| | | | | mammary carcinomas, | |
| | | | | H/N SCC, NSCLC, | |
| | | | | sarcoma | |
| DAM-6, -10 | A2 | FLWGPRAYA | (SEQ ID NO:32) | Fleischhauer et al | Melanoma, skin |
| | | | 1998 | tumors, mammary and | |
| | | | | ovarian carcinomas | |
| | | | | (Lurquin et al., 1997)—lung | |
| | | | | carcinoma | |
| | | | | (Dabovic et al., 1995; | |
| | | | | Lurquin et al., 1997)—seminomas | |
| | | | | (Dabovic et | |
| | | | | al., 1995) | |
| GAGE-1, -2, | Cw6 | YRPRPRRY | (SEQ ID NO:33) | Van den Eynde et | Melanoma, sarcoma, |
| -8 | | | al. 1995 | NSCLC, SCLC, | |
| | | | De Backer et al. | mesothelioma, | |
| | | | 1999 | sarcoma, seminoma, | |
| | | | | leukemias, lymphomas, | |
| | | | | H/N tumors, bladder, | |
| | | | | esophageal, mammary, | |
| | | | | colon, prostate | |
| | | | | carcinomas | |
| GAGE-1, -4, | A29 | YYWPRPRRY | (SEQ ID NO:34) | De Backer et al | Melanomas, H/N |
| -5, -6, -7B | | | 1999 | tumors, leukemias, | |
| | | | | esophageal, lung and | |
| | | | | bladder carcinomas | |
| NA88-A | B13 | MTQGQHFLQKV | (SEQ ID NO:35) | Moreau-Aubrey, et | Melanoma |
| | | | al. 2000 | | |
| NY-ESO-1 | A2 | SLLMWITQCFL | (SEQ ID NO:36) | Jäger et al. 1998 | Melanoma, sarcoma, |
| | | | | B-lymphomas,hepatoma, | |
| NY-ESO-1a | A2 | SLLMWITQC | (SEQ ID NO:37) | Jäger et al. 1998 | H/N tumors, bladder, |
| | | | | lung, prostate, ovarian, | |
| | | | | thyroid and breast | |
| (CAG-3) | A2 | QLSLLMWIT | (SEQ ID NO:38) | Jäger et al. 1998 | carcinoma (Chen et al., |
| | | | | 1997) | |
| | A31 | ASGPGGGAPR (SEQ ID NO:39) | Wang et al. 1998b | | |

^{a}Tissue distribution among tumors as described in the given references when different from the paper first reporting the sequence of the epitope.

The following table is from Renkvist et al. (2001).

TABLE 2

Class I HLA-restricted melanocyte differentiation antigens. These
antigens can only be expressed in normal and neoplastic cells of the same
lineage (namely melanocytes, skin, retina, peripheral ganglia) or in normal
cells of the prostate gland.

| | | | |
|---|---|---|---|
| Gene | HLA allele | Peptide epitope | Authors |

| | | | | |
|---|---|---|---|---|
| MART-1/Melan-A^{a} | A2 | AAGIGILTV | (SEQ ID NO:40) | Coulie et al. 1994 |
| | | | | Kawakami et al. 1994a |
| | A2 | EAAGIGILTV | (SEQ ID NO:41) | Schneider et al. 1998 |
| | A2 | ILTVILGVL | (SEQ ID NO:42) | Castelli et al. 1995 |
| | B45 | AEEAAGIGIL | (SEQ ID NO:43) | Schneider et al. 1998 |
| | B45 | AEEAAGIGILT | (SEQ ID NO:44) | Schneider et al. 1998 |
| MCIR | A2 | TILLGIFFL | (SEQ ID NO:45) | Salazar-Onfray et al. 1997 |
| | A2 | FLALIICNA | (SEQ ID NO:46) | Salazar-Onfray et al. 1997 |
| Gp100 | A2 | KTWGQYWQV | (SEQ ID NO:47) | Bakker et al. 1995 |
| | A2 | AMLGTHTMEV | (SEQ ID NO:48) | Tsai et al. 1997 |
| | A2 | MLGTHTMEV | (SEQ ID NO:49) | Tsai et al. 1997 |
| | A2 | SLADTNSLAV | (SEQ ID NO:50) | Tsai et al. 1997 |
| | A2 | ITDQVPFSV | (SEQ ID NO:51) | Kawakami et al. 1995 |
| | A2 | LLDGTATLRL | (SEQ ID NO:52) | Kawakami et al. 1994b |
| | A2 | YLEPGPVTA | (SEQ ID NO:53) | Cox et al. 1994 |
| | A2 | VLYRYGSFSV | (SEQ ID NO:54) | Kawakami et al. 1995 |
| | A2 | RLMKQDFSV | (SEQ ID NO:55) | Kawakami et al. 1998 |
| | A2 | FLPRIFCSC | (SEQ ID NO:56) | Kawakami et al. 1998 |
| | A3 | LIYRRRLMK | (SEQ ID NO:57) | Kawakami et al. 1998 |
| | A3 | ALNFPGSQK | (SEQ ID NO:58) | Kawashima et al. 1998 |
| | A3 | SLIYRRRLMK | (SEQ ID NO:59) | Kawashima et al. 1998 |
| | A3 | ALLAVGATK | (SEQ ID NO:60) | Skipper et al. 1996 |
| | A24 | VYFFLPDHL | (SEQ ID NO:61) | Robbins et al. 1997 |
| | Cw8 | SNDGPTLI | (SEQ ID NO:62) | Castelli et al. 1999 |
| PSA | A1 | VSHSFPHPLY | (SEQ ID NO:63) | Corman et al. 1998 |
| | A2 | FLTPKKLQCV | (SEQ ID NO:64) | Correale et al. 1997 |
| | A2 | VISNDVCAQV | (SEQ ID NO:65) | Correale et al. 1997 |
| PSM | A1 | HSTNGVTRIY | (SEQ ID NO:66) | Corman et al. 1998 |
| Tyrosinase | A1 | KCDICTDEY | (SEQ ID NO:67) | Kittlesen et al. 1998 |
| | A1 | SSYVIPIGTY | (SEQ ID NO:68) | Kawakami et al. 1998 |
| | A2 | YMDGTMSQV | (SEQ ID NO:69) | Wölfel et al. 1994 |
| | A2 | MLLAVLYCL | (SEQ ID NO:70) | Wölfel et al. 1994 |
| | A24 | AFLPWHRLF | (SEQ ID NO:71) | Kang et al. 1995 |
| | B44 | SETWRDIDF | (SEQ ID NO:72) | Brichard et al. 1996 |
| TRP-1 (or gp75) | A31 | MSLQRQFLR | (SEQ ID NO:73) | Wang et al. 1996b |
| TRP-2 | A2 | SVYDFFVWL | (SEQ ID NO:74) | Parkhurst et al. 1998 |
| | A2 | TLDSQVMSL | (SEQ ID NO:75) | Noppen et al. 2000 |
| | A31 | LLGPGRPYR | (SEQ ID NO:76) | Wang et al. 1996a |
| | A33 | LLGPGRPYR | (SEQ ID NO:77) | Wang et al. 1998a |
| | Cw8 | ANDPIFVVL | (SEQ ID NO:78) | Castelli et al. 1999 |

^{a}TWO different groups simultaneously discovered this gene and gave it two different names, MART-1 and Melan-A respectively

The following table is from Renkvist et al. (2001).

TABLE 3

Class I HLA-restricted widely expressed antigens

Tissue distribution

| | | | | | |
|---|---|---|---|---|---|
| Gene | HLA | Peptide epitope | Tumors | Normal Tissues | Reference |

| | | | | | | |
|---|---|---|---|---|---|---|
| ART-4 | A24 | AFLRHAAL | (SEQ ID NO:79) | SCC, SCLC, | Testis, placenta, | Kawano et al. |
| | | DYPSLSATDI | (SEQ ID NO:80) | H/N tumors, | fetal liver | 2000 |
| | | | leukemia, lung, | | | |
| | | | esophageal, | | | |
| | | | gastric, | | | |
| | | | cervical, | | | |
| | | | endometrial, | | | |
| | | | ovarian and | | | |
| | | | breast | | | |
| | | | carcinomas | | | |
| CAMEL | A2 | MLMAQEALAFL | (SEQ ID NO:81) | Melanoma | Testis, placenta, | Aarnoudse et |
| | | | | heart, skeletal | al. 1999 | |
| | | | | muscle, pancreas | | |
| CEA | A2 | YLSGANLNL | (SEQ ID NO:82) | Melanoma | Testis, placenta, | Tsang et al. |
| | | (CAP-1)^{a} | heart, skeletal | 1995 | | |
| | | | muscle, pancreas | | | |
| CEA | A3 | HLFGYSWYK | (SEQ ID NO:83) | Colon, rectum, | Gastrointestinal | Kawashima et |
| | | | pancreas, | embryonic tissue | al. 1999 | |
| | | | gastric, breast | | | |
| | | | and lung | | | |
| | | | carcinomas | | | |
| Cyp-B | A24 | KFHRVIKDF | (SEQ ID NO:84) | Lung adenocarcinoma, | Ubiquitously | Gomi et al. |
| | | | T cell | expressed in | 1999 | |
| | | DFMIQGGDF | (SEQ ID NO:85) | leukemia, | normal tissues | |
| | | | lymphosarcoma— | | | |
| | | | bladder, | | | |
| | | | ovarian, uterine | | | |
| | | | and esophagela | | | |
| | | | SCC, | | | |
| HER2/neu | A2 | KIFGSLAFL | (SEQ ID NO:86) | Melanoma—ovarian | Epithelial cells | Risk et al. |
| | | | and | | 1995 | |
| | | | breast | | | |
| | | | carcinomas | | | |
| HER2/neu | A2 | IISAVVGIL | (SEQ ID NO:87) | Melanoma, ovarian, | Epithelial cells | Peoples et al. |
| | | | pancreatic | | 1995 | |
| | | | and | | (Pieper et al., | |
| | | | breast | 1999)^{b} | | |
| | | | carcinomas | | | |
| HER2/neu | A2 | RLLQETELV | (SEQ ID NO:88) | Melanoma, | Epithelial cells | Kono et al. |
| | | | ovarian, | | 1998 | |
| | | | gastric, | (Pieper et al., | | |
| | | | pancreatic | | 1999) | |
| | | | and | | | |
| | | | breast | | | |
| | | | carcinomas | | | |
| HER 2/neu | A2 | VVLGVVFGI | (SEQ ID NO:89) | Melanoma, | Epithelial cells | Rongcun et al. |
| | | | ovarian, | | 1999 | |
| | | ILHNGAYSL | (SEQ ID NO:90) | gastric, | | |
| | | | pancreatic | | | |
| | | YMIMVKCWMI | (SEQ ID NO:91) | (Pieper et al., | | |
| | | | 1999) and | | | |
| | | | breast | | | |
| | | | carcimonas | | | |
| HER2/neu | A3 | VLRENTSPK | (SEQ ID NO:92) | Melanoma, | Epithelial cells | Kawashima et |
| | | | ovarian, | | al. 1999 | |
| | | | gastric, | | (Pieper et al., | |
| | | | pancreatic | 1999) | | |
| | | | and | | | |
| | | | breast | | | |
| | | | carcinomas | | | |
| HTERT^{c} | A2 | ILAKFLHWL | (SEQ ID NO:93) | Lung, and | Hematopoietic | Vonderheide |
| | | | ovarian | stem cells and | et al.1999 | |
| | | | carcinomas—multiple | progenitors; | | |
| | | | myeloma, | germinal center | | |
| | | | melanoma, | cells; basal | | |
| | | | sarcoma, acute | keratinocytes; | | |
| | | | leukemias, | gonadal cells; | | |
| | | | non-Hodgkin's | certain | | |
| | | | lymphomas | proliferating | | |
| | | | | epithelial cells | | |
| HTRT^{c} | A2 | ILAKFLHWL | (SEQ ID NO:94) | Lung, prostate | Circulating B | Minev et al. |
| | | RLVDDFLLV | (SEQ ID NO:95) | and ovarian | cells; germinal | 2000 |
| | | | carcinomas, | center B cells; | | |
| | | | multiple | thymocytes; | | |
| | | | myeloma, | CD34+ progenitor | | |
| | | | melanoma, | hemopoietic | | |
| | | | sarcoma, acute | cells | | |
| | | | leukemias, | | | |
| | | | non-Hodgkin's | | | |
| | | | lymphomas | | | |
| iCE | B7 | SPRWWPTCL | (SEQ ID NO:96) | RCC | Kidney, colon, | Ronsin et al. |
| | | | | small intestine, | 1999 | |
| | | | | liver, heart, | | |
| | | | | pituitary gland, | | |
| | | | | adrenal gland, | | |
| | | | | prostate, stomach | | |
| MUCI | A11 | STAPPAHGV | (SEQ ID NO:97) | Breast and | None^{d} | Domenech et |
| | | | ovarian | | al. 1995 | |
| | | | carcinomas, | | | |
| | | | multiple | | | |
| | | | myeloma, B-cell | | | |
| | | | carcinoma, | | | |
| | | | multiple | | | |
| | | | myeloma | | | |
| MUCI | A2 | STAPPVHNV | (SEQ ID NO:98) | Breast and | None^{d} | Brossart et al. |
| | | | ovarian | | 1999 | |
| | | | carcinoma, | | | |
| | | | multiple | | | |
| | | | myeloma, B-cell | | | |
| | | | lymphoma | | | |
| MUC2 | A2 | LLNQLQVNL | (SEQ ID NO:99) | Ovary, | Colon, small | Bohm et at. |
| | | MLWGWREHV | (SEQ ID NO:100) | pancreas and | intestine, | 1998 |
| | | | breast | bronchus, cervix | | |
| | | | mucinous | and gall bladder | | |
| | | | tumors, colon | | | |
| | | | carcinoma of | | | |
| | | | non-mucinous | | | |
| | | | type | | | |
| PRAME | A24 | LYVDSLFFL | (SEQ ID NO:101) | Melanoma, | Testis, | Ikeda et al. |
| | | | H/N and lung | endometrium, | 1997 | |
| | | | SCC, NSCLC | ovary, adrenals, | | |
| | | | (van Baren et | kidney, brain, | | |
| | | | al.,1998), | skin | | |
| | | | RCC, | | | |
| | | | adenocarcinoma, | | | |
| | | | sarcoma, | | | |
| | | | leukemias | | | |
| P15 | A24 | AYGLDFYIL | (SEQ ID NO:102) | Melanoma | Testis, spleen, | Robbins et al |
| | | | | thymus, liver, | 1995 | |
| | | | | kidney, adrenal | | |
| | | | | tissue, lung | | |
| | | | | tissue, retinal | | |
| | | | | tissue | | |
| RUI | B51 | VPYGSFKHV | (SEQ ID NO:103) | Melanoma, | Testis, kidney, | Morel et al. |
| | | | renal and | heart, skin, brain, | 2000 | |
| | | | bladder | ovary, liver, lung, | | |
| | | | carcinomas | lymphocytes, | | |
| | | | | thymus, | | |
| | | | | fibroblasts | | |
| RU2 | B7 | LPRWPPPQL | (SEQ ID NO:104) | elanoma, | Testis, kidney, | Van den |
| | | | sarcomas | liver, urinary | Eynde et al. | |
| | | | leukemia—brain, | bladder | 1999 | |
| | | | esophageal and | | | |
| | | | H/N tumors—renal, | | | |
| | | | colon, | | | |
| | | | thyroid, | | | |
| | | | mammary, | | | |
| | | | bladder, | | | |
| | | | prostatic and | | | |
| | | | lung | | | |
| | | | carcinomas | | | |
| SART-1 | A24 | EYRGFTQDF | (SEQ ID NO:105) | Esophageal, | Testis, fetal liver | Kikuchi et al |
| | | | H/N and lung | | 1999 | |
| | | | SCC—adenocarcinom | | | |
| | | | a, uterine | | | |
| | | | cancer | | | |
| SART-1 | A*2 | KGSGKMKTE | (SEQ ID NO:106) | Esophageal, | Testis, fetal liver | Shichijo et al. |
| | 601 | | H/N and lung | | 1998 | |
| | | | SCC, | | | |
| | | | adenocarcinoma, | | | |
| | | | uterine | | | |
| | | | cancer | | | |
| SART-3 | A24 | VYDYNCHVDL | (SEQ ID NO:107) | H/N, | Lymphoid cells, | Yang et al |
| | | AYIDFEMKI | (SEQ ID NO:108) | esophageal and | fibroblasts, testis, | 1999 |
| | | | lung SCC, | fetal liver | | |
| | | | adenocarcinoma, | | | |
| | | | leukemia, | | | |
| | | | melanoma | | | |
| WT1 | A2 | RMFPNAPYL | (SEQ ID NO:109) | Gastric, colon, | Kidney, ovary, | Oka et al. |
| | | | lung, breast, | testis, spleen | 2000 | |
| | | | ovary, uterine, | | | |
| | | | thyroid and | | | |
| | | | hepatocellular | | | |
| | | | carcinomas—leukemia | | | |
| | | | (including | | | |
| | | | AML, ALL and | | | |
| | | | CML) | | | |

^{a}CAP-1 is an alternative name of this peptide
^{b}Tissue distribution among tumors as described in the given references when different from the paper first reporting the sequence of the epitope
^{c}Telomerase is expressed in most human tumors: those listed were shown to be susceptible to lysis by cytotoxic T lymphocytes
^{d}All epithelial tissues express mucin-like hyperglycosylated molecules

The following table is from Renkvist et al. (2001).

TABLE 4

CLASS I HLA-RESTRUCTED TUMOR-SPECIFIC ANTIGENS, INCLUDING BOTH UNIQUE
(CDK-4, MUM-2, β-CATENIN, HLA-A2 -R1701, ELF2 M, MYOSIN-M, CASPASE-8, KIAA0205, HSP70-2M)
AND SHARED (CAMEL, TRP-2/INT2, GNT-V 250, ANTIGENS

Tissue distribution

| | | | | | |
|---|---|---|---|---|---|
| Gene | HLA allele | Peptide epitope | Tumors | Normal tissues | Reference |

| | | | | | | |
|---|---|---|---|---|---|---|
| AFP | A2 | GVALQTMKQ | (SEQ ID NO:110) | Hepatocellular | Fetal liver | Butterfield et al. |
| | | | | carcinoma | | 1999 |
| β-Catenin/m | A24 | SYLDSGIHF | (SEQ ID NO:111) | Melanoma | None | Robbins et al. 1996 |
| Caspase-8/m | A2 | FPSDSWCYF | (SEQ ID NO:112) | H/N tumors | None | Mandruzzato et al. |
| | | | | | | 1997 |
| CDK-4/m | A2 | ACDPHSGHFV | (SEQ ID NO:113) | Melanoma | None | Wõfel et al. 1995 |
| ELF2 M | A68 | ETVSEQSNV | (SEQ ID NO:114) | Lung SCC | None | Hogan et al. 1998 |
| GnT-V | A2 | VLPDVFIRC(V)^{a} | (SEQ ID NO:115) | Melanoma, brain tumors, | Breast and | Guilloux et al. |
| | | | | sarcoma | brain (low | 1996 |
| | | | | | expression) | |
| G250 | A2 | HLSTAFARV | (SEQ ID NO:116) | RCC, colon, ovarian and | None | Vissers et al. 1991 |
| | | | | cervical carcinomas | | |
| HSP70-2M | A2 | SLFEGIDIY | (SEQ ID NO:117) | RCC, melanoma, | None | Gaudin et al. 1999 |
| | | | | neuroblastoma | | |
| HA-A*0201-R1701 | A2 | CVEWLRIYLENGK | (SEQ ID NO:118) | RCC | None | Br\overline{a}ndle et al. 1996 |
| HST-2 | A31 | YSWMDISCWI | (SEQ ID NO:119) | Gastric signet cell | None | Suzuki et al. 1999 |
| | | | | carcinoma | | |
| KIAA0205 | B44*03 | AEPINIQTV | (SEQ ID NO:120) | Bladder cancer | None | Gueguen et al. |
| | | | | | | 1998 |
| MUM-1 | B44 | EEKLIVVLF | (SEQ ID NO:121) | Melanoma | None | Coulie et al. 1995 |
| MUM-2 | B44 | SELFRSGLDY | (SEQ ID NO:122) | Melanoma | None | Coulie et al. 1995 |
| MUM-2 | Cw6 | FRSGLDSYV | (SEQ ID NO:123) | Melanoma | None | Chiari et al. 1999 |
| MUM-3 | A28 | EAFIQPITR | (SEQ ID NO:124) | Melanoma | None | Baurain et al. 2000 |
| Myasin/m | A3 | KINKNPKYK | (SEQ ID NO:125) | Melanoma | None | Zorn and Hercend, |
| | | | | | | 1999a |
| RAGE | B7 | SPSSNRIRNT | (SEQ ID NO:126) | Melanoma, sarcomas, | Retina | Gaugler et al. 1996 |
| | | | | mesotheliomas, H/N | only | |
| | | | | tumors, bladder, renal, | | |
| | | | | colon and mammary | | |
| | | | | carcinomas | | |
| SART-2 | A24 | DYSARWNEI | (SEQ ID NO:127) | H/N and lung SCC, lung | None | Nakao et al. 2000 |
| | | | | adenocarcinoma, RCC, | | |
| | | | | melanoma, brain tumors, | | |
| | | | | esophageal and uterine | | |
| | | | | cancers | | |
| TRP-2/INT2 | A68 | AYDFLYNYL | (SEQ ID NO:128) | Melanoma | None | Lupetti et al. 1998 |
| | | SYTRLFLIL | (SEQ ID NO:129) | | | |
| | | EVISCKLIKR | (SEQ ID NO:130) | | | |
| 707-AP | A2 | RVAALARDA | (SEQ ID NO:131) | Melanoma | None^{b} | Morioka et al |
| | | | | | | 1995 |

^{a}VLPDVFIRC(V) = nonamer and decamer peptides are both recognized by CTLs
^{b}This antigen is not expressed in normal cells but, as the tissue of the testis was not tested, it will not become clear to which category the antigen may belong until more information is available

The following table is from Renkvist et al. (2001).

TABLE 5

CLASS II HLA-RESTRICTED ANTIGENS

Tissue distribution

| | | | | | |
|---|---|---|---|---|---|
| | HLA | | | Normal | |
| Gene | allele | Peptide epitope | Tumors | tissues | Reference |

Epitopes from normal protein antigens

| | | | | | | |
|---|---|---|---|---|---|---|
| Amexin II | DRB*0401 | DVPKWISIMTERSVPH | (SEQ ID NO:132) | Melanoma | Not done | Li et al. |
| | | | | | 1998 | |
| Gp100 | DRB1*0401 | WNRQLYPEWTEAQRL | (SEQ ID NO:133) | (Melanoma | Melanocytes | Li et al. |
| | | D | | 1998 | | |
| MAGE-1, - 2, | DRB*1301 | LLKYRARIPVTKAE | (SEQ ID NO:134) | Melanoma, lung and | Testis, | Chaux et |
| -3, -6 | DRB*1302 | | breast carcinomas, | placenta | al. 1999a | |
| | | | H/N SCC | | | |
| MAGE-3 | DR*11-1 | TSYVKVLHHMVKISG | (SEQ ID NO:135) | Melanoma, lung and | Testis, | Manici et |
| | | | breast carcinomas, | placenta | al. 1999 | |
| | | | H/N SCC | | | |
| MAGE-3 | DRB*1301 | AELVHFLLLKYRAR | (SEQ ID NO:136) | Melanoma, lung and | Testis, | Chaux et |
| | DRB*1302 | | breast carcinomas, | placenta | al. 1999b | |
| | | | H/N SCC | | | |
| MART-1/ | DRB1*0401 | RNGYRALMDKS | (SEQ ID NO:137) | Melanoma | Melanocytes | Zarour et |
| Melan-A | | LHVGTQCALTRR | | | al. 2000 | |
| MUCI | DR3 | PGSTAPPAHGVT | (SEQ ID NO:138) | Breast and ovarian | None^{a} | Hitbold et |
| | | | cancers; multiple | | al. 1998 | |
| | | | myeloma, B-cell | | | |
| | | | lymphoma | | | |
| NY-ESO-1 | DRB4*0101 | VLLKEFTVSG | (SEQ ID NO:139) | Melanoma, B-lymphoma, | Testis | Zeng et al. |
| | | | lymphoma, | | 2000 | |
| | | | hepatoma (Chen et | | | |
| | | | al., 1997)^{b}, sarcoma, | | | |
| | | | H/N tumors,—bladder, | | | |
| | | | lunch, | | | |
| | | | prostate, ovarian, | | | |
| | | | thyroid and breast | | | |
| | | | carcinomas | | | |
| NY-ESO-1 | DRB4*0101- | PLPVPGVLLKEFTVSN | (SEQ ID NO:140) | B-Lymphoma, | Testis | Jäger et |
| | 0103 | GIVLLKEFTVSGNILTI | melanoma, sarcoma, | | al, 2000 | |
| | | RLTAADHRQLQLSISS | H/N tumors, | | | |
| | | CLQQL | hepatoma (Chen et | | | |
| | | | al., 1997)—bladder, | | | |
| | | | lung, prostate, | | | |
| | | | ovarian, thyroid and | | | |
| | | | breast carcinomas | | | |
| PSA | DR4 | ILLGRMSLFMPEDTG | (SEQ ID NO:141) | Melanoma | Melanocytes | Corman et |
| | | SLFHPEDTGQVFQ | | | al. 1998 | |
| | | QVFQVSHSFPHPLYD | | | | |
| | | NDLMLLRLSEPAELT | | | | |
| | | KKLQCVQLHVISM | | | | |
| | | GVLQGITSMGSEPCA | | | | |
| Tyrosinase | DRB1*0401 | QNILLSNAPLGPQFP | (SEQ ID NO:142) | Melanoma | Melanocytes | Topalian |
| | | DYSYLQDSDPDSFQD | | | et al. 1994 | |
| | | SYLQDSDPDSFQD | | | Topalian | |
| | | | | | et al. 1996 | |
| Tyrosinase | DRB1*1501 | RHRPLQEVYPEANAPI | (SEQ ID NO:143) | Melanoma | Melanocytes | Kobayashi |
| | | GHNRE | | | et al. | |
| | | | | | 1998a | |
| Tyrosinase | DRB1*0405 | EIWRDIDFAHE | (SEQ ID NO:144) | Melahoma | Melanocytes | Kobayashi |
| | | | | | et al. | |
| | | | | | 1998b | |

Epitopes from mutated protein antigens

| | | | | | | |
|---|---|---|---|---|---|---|
| HPV-E7 | DR*0401 | LFMDTLSFVCPLC | (SEQ ID NO:145) | Cervical carcinomas | None | Höhn et |
| | | | | | al. 1999 | |
| | DR*0407 | LFMDSLNFVCPWC | (SEQ ID NO:146) | | | |
| CDC27/m | DRB1*0401 | FSWAMDLDPKGA | (SEQ ID NO:147) | Melanoma | None | Wang et |
| | | | | | al. | |
| | | | | | 1999a | |
| TPI/m | DRB1*0101 | GELIGILNAAKVPAD | (SEQ ID NO:148) | Melanoma | None | Pieper |
| | | | | | et al. | |
| | | | | | 1999 | |

^{a}All epithelial tissues express highly glycosilated mucins whereas tumor cells often show hypoglycosilated mucins with a normal protein sequence.
^{b}Tissue distribution among tumors as described in the given references when different from the paper first reporting the sequence of the epitope.

Ex vivo Culture of Dendritic Cells

In some embodiments of the present invention, a mutant fibronectin, either polynucleotide or polypeptide in form, is administered to an immune effector cell, such as a dendritic cell. Such manipulation, in some embodiments, requires ex vivo culture of the immune effector cells. As an exemplary embodiment, procedures for a dendritic cell are described herein.

A procedure for ex vivo expansion of hematopoietic stem and progenitor cells is described in U.S. Pat. No. 5,199,942, incorporated herein by reference. Other suitable methods are known in the art. Briefly, ex vivo culture and expansion comprises: (1) collecting CD34+ hematopoietic stem and progenitor cells from a patient from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used.

Stem or progenitor cells having the CD34 marker constitute only about 1% to 3% of the mononuclear cells in the bone marrow. The amount of CD34+ stem or progenitor cells in the peripheral blood is approximately 10- to 100-fold less than in bone marrow. Cytokines such as flt3-L may be used to increase or mobilize the numbers of dendritic cells in vivo. Increasing the quantity of an individual's dendritic cells may facilitate antigen presentation to T cells for antigen(s) that already exists within the patient, such as a disease antigen, such as a tumor antigen, or a bacterial or viral antigen. Alternatively, cytokines may be administered prior to, concurrently with or subsequent to administration of an antigen to an individual for immunization purposes.

Peripheral blood cells are collected using apheresis procedures known in the art. (See, for example, Bishop et al., 1994). Briefly, peripheral blood progenitor cells (PBPC) and peripheral blood stem cells (PBSC) are collected using conventional devices, for example, a Haemonetics Model V50 apheresis device (Haemonetics, Braintree, Mass.). Four-hour collections are performed typically no more than five times weekly until approximately 6.5×108 mononuclear cells (MNC)/kg are collected. The cells are suspended in standard media and then centrifuged to remove red blood cells and neutrophils. Cells located at the interface between the two phases (the buffy coat) are withdrawn and resuspended in HBSS. The suspended cells are predominantly mononuclear and a substantial portion of the cell mixture are early stem cells.

A variety of cell selection techniques are known for identifying and separating CD34+ hematopoietic stem or progenitor cells from a population of cells. For example, monoclonal antibodies (or other specific cell binding proteins) can be used to bind to a marker protein or surface antigen protein found on stem or progenitor cells. Several such markers or cell surface antigens for hematopoietic stem cells (i.e., flt-3, CD34, My-10, and Thy-1) are known in the art, as are specific binding proteins therefore (see for example, U.S. Ser. No. 08/539,142, filed Oct. 4, 1995).

In one method, antibodies or binding proteins are fixed to a surface, for example, glass beads or flask, magnetic beads, or a suitable chromatography resin, and contacted with the population of cells. The stem cells are then bound to the bead matrix. Alternatively, the binding proteins can be incubated with the cell mixture and the resulting combination contacted with a surface having an affinity for the antibody-cell complex. Undesired cells and cell matter are removed providing a relatively pure population of stem cells. The specific cell binding proteins can also be labeled with a fluorescent label, e.g., chromophore or fluorophore, and the labeled cells separated by sorting. Preferably, isolation is accomplished by an immunoaffinity column.

Immunoaffinity columns can take any form, but usually comprise a packed bed reactor. The packed bed in these bioreactors is preferably made of a porous material having a substantially uniform coating of a substrate. The porous material, which provides a high surface area-to-volume ratio, allows for the cell mixture to flow over a large contact area while not impeding the flow of cells out of the bed. The substrate should, either by its own properties, or by the addition of a chemical moiety, display high-affinity for a moiety found on the cell-binding protein. Typical substrates include avidin and streptavidin, while other conventional substrates can be used.

In one useful method, monoclonal antibodies that recognize a cell surface antigen on the cells to be separated are typically further modified to present a biotin moiety. The affinity of biotin for avidin thereby removably secures the monoclonal antibody to the surface of a packed bed (see Berenson et al., 1986). The packed bed is washed to remove unbound material, and target cells are released using conventional methods. Immunoaffinity columns of the type described above that utilize biotinylated anti-CD34 monoclonal antibodies secured to an avidin-coated packed bed are described for example, in WO 93/08268.

An alternative means of selecting the quiescent stem cells is to induce cell death in the dividing, more lineage-committed, cell types using an antimetabolite such as 5-fluorouracil (5-FU) or an alkylating agent such as 4-hydroxycyclophosphamide (4-HC). The non-quiescent cells are stimulated to proliferate and differentiate by the addition of growth factors that have little or no effect on the stem cells, causing the non-stem cells to proliferate and differentiate and making them more vulnerable to the cytotoxic effects of 5-FU or 4-HC. (See Berardi et al., 1995), which is incorporated herein by reference.)

Isolated stem cells can be frozen in a controlled rate freezer (e.g., Cryo-Med, Mt. Clemens, Mich.), then stored in the vapor phase of liquid nitrogen using dimethylsulfoxide as a cryoprotectant. A variety of growth and culture media can be used for the growth and culture of dendritic cells (fresh or frozen), including serum-depleted or serum-based media. Useful growth media include RPMI, TC 199, Iscoves modified Dulbecco's medium (Iscove et al., 1978), DMEM, Fischer's, alpha medium, NCTC, F-10, Leibovitz's L-15, MEM and McCoy's.

Particular nutrients present in the media include serum albumin, transferrin, lipids, cholesterol, a reducing agent such as 2-mercaptoethanol or m6monothioglycerol, pyruvate, butyrate, and a glucocorticoid such as hydrocortisone 2-hemisuccinate. More particularly, the standard media includes an energy source, vitamins or other cell-supporting organic compounds, a buffer such as HEPES, or Tris, that acts to stabilize the pH of the media, and various inorganic salts. A variety of serum-free cellular growth media is described in WO 95/00632, which is incorporated herein by reference.

The collected CD34+ cells are cultured with suitable cytokines, for example, as described herein, and in U.S. Ser. No. 08/539,142. CD34+ cells then are allowed to differentiate and commit to cells of the dendritic lineage. These cells are then further purified by flow cytometry or similar means, using markers characteristic of dendritic cells, such as CD1a, HLA DR, CD80 and/or CD86. The cultured dendritic cells are exposed to an antigen, for example, a tumor antigen or an antigen derived from a pathogenic or opportunistic organism, allowed to process the antigen, and then cultured with an amount of a CD40 binding protein to activate the dendritic cell. Alternatively, the dendritic cells are transfected with a gene encoding an antigen, and then cultured with an amount of a CD40 binding protein to activate the antigen-presenting dendritic cells.

Identifying T Cell Epitopes

In one specific embodiment of the present invention, to identify peptides that are recognized by either CD4+ or CD8+ T cells, a series of overlapping peptides (for example, about 9-13 amino acids) will be made based on the predicted amino acid sequence from a gene of interest, or an altered sequence thereof. T cells recognize a peptide bound to the MHC class I or II molecules. The synthetic peptides are then tested for their ability to stimulate cytokine secretion from T cells when pulsed onto MHC matched antigen presenting cells (APC) such as EBV transformed B cells or dendritic cells. Once positive peptides are identified, a series of truncations are made at the N- and C-terminus of a peptide such that a minimal length of a T-cell peptide is defined.

Kits

Any of the compositions described herein may be comprised in a kit. In a non-limiting example, a composition comprising a mutant fibronectin may be comprised in a kit. In a specific embodiment, the composition is a vaccine. In another specific embodiment, the mutant fibronectin and an additional tumor antigen are housed in a kit and an immune effector cell, such as a dendritic cell, is provided elsewhere, such as derived from the patient being treated with the kit component(s). The kits may thus comprise, in suitable container means, a mutant fibronectin, a dendritic cell, and/or another tumor antigen, such as may be comprised on or with a vector. In the embodiment wherein the kit is for cancer treatment, the kit contains a tumor antigen associated with a particular cancer.

The kits may comprise suitably aliquoted dendritic cells and/or related components of the invention compositions of the present invention, whether labeled or unlabeled, as may be used to prepare a standard curve for a detection assay. Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the kit components in their containers in close confinement for commercial sale. Such containers may include injection or blow-molded plastic containers into which the desired vials are retained.

The kits will generally contain, in suitable container means, a pharmaceutically acceptable formulation of the mutant fibronectin, an immune effector cell, such as a dendritic cell, and/or an additional tumor antigen. The kit may have a single container means, and/or it may have distinct container means for each compound. When the components of the kit are provided in one and/or more liquid solutions, the liquid solution is an aqueous solution, with a sterile aqueous solution being particularly preferred. The compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit. However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

Irrespective of the number and/or type of containers, the kits of the invention may also comprise, and/or be packaged with, an instrument for assisting with the injection/administration and/or placement of the ultimate composition(s), or a precursor thereof, within the body of an animal. Such an instrument may be a syringe, pipette, forceps, and/or any such medically approved delivery vehicle.

Immunotherapeutic Agents

The disclosures presented herein have significant relevance to immunotherapy of human diseases and disorders, including cancer. In using the immunotherapeutic compositions of the present invention in treatment methods for cancer, other standard treatments also may be employed, such as radiotherapy or chemotherapy. However, in specific embodiments additional immunotherapy methods may also be used. Some immunotherapies of cancer are described in the following sections and may be used or produced with the methods and compositions of the present invention.

An immunotherapeutic agent generally relies on the use of immune effector cells and molecules to target and destroy cancer cells. In one aspect of immunotherapy, the tumor cell must bear some marker that is amenable to targeting, i.e., is not present on the majority of other cells. Many tumor markers exist and any of these may be suitable for targeting in the context of the present invention.

Immune Stimulators

A specific aspect of immunotherapy is to use an immune stimulating molecule as an agent, or more preferably in conjunction with another agent, such as, for example, a cytokine such as, for example, IL-2, IL-4, IL-12, GM-CSF, tumor necrosis factor; interferons alpha, beta, and gamma; F42K and other cytokine analogs; a chemokine such as, for example, MIP-1, MIP-1beta, MCP-1, RANTES, IL-8; or a growth factor such as, for example, FLT3 ligand.

One particular cytokine contemplated for use in the present invention is tumor necrosis factor. Tumor necrosis factor (TNF; Cachectin) is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon- [symbol] also has been found to possess anti-cancer activity.

Another cytokine specifically contemplated is interferon alpha. Interferon alpha has been used in treatment of hairy cell leukemia, Kaposi's sarcoma, melanoma, carcinoid, renal cell cancer, ovary cancer, bladder cancer, non-Hodgkin's lymphomas, mycosis fungoides, multiple myeloma, and chronic granulocytic leukemia.

Active Immunotherapy

In active immunotherapy, an antigenic peptide, polypeptide or protein, or an autologous or allogenic tumor cell composition or “vaccine” is administered, generally with a distinct bacterial adjuvant (Ravindranath & Morton, 1991; Morton & Ravindranath, 1996; Morton et al., 1992; Mitchell et al., 1990; Mitchell et al., 1993). In melanoma immunotherapy, those patients who elicit high IgM response often survive better than those who elicit no or low IgM antibodies (Morton et al., 1992). IgM antibodies are often transient antibodies and the exception to the rule appears to be anti-ganglioside or anticarbohydrate antibodies.

Adoptive Immunotherapy

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989). To achieve this, one would administer to an animal, or human patient, an immunologically effective amount of activated lymphocytes in combination with an adjuvant-incorporated anigenic peptide composition as described herein. The activated lymphocytes will most preferably be the patient's own cells that were earlier isolated from a blood or tumor sample and activated (or “expanded”) in vitro. This form of immunotherapy has produced several cases of regression of melanoma and renal carcinoma.

Enhancement of an Immune Response

The present invention includes a method of enhancing the immune response in a subject comprising the steps of contacting one or more lymphocytes with an antigenic composition, wherein the antigen is presented by an immune system cell, such as the dendritic cells of the present invention, and in a specific embodiment comprises a mutant fibronectin, or an immunologically functional equivalent thereof. As used herein, an “antigenic composition” may comprises an antigen (e.g., a peptide). In certain embodiments, the antigenic composition is conjugated to or comprises an HLA anchor motif amino acids.

In other embodiments, the compositions of the present invention are in a mixture that comprises an additional immunostimulatory agent or nucleic acids encoding such an agent. Immunostimulatory agents include but are not limited to an additional antigen, an immunomodulator, an antigen presenting cell and/or an adjuvant. In other embodiments, one or more of the additional agent(s) is covalently bonded to the antigen or an agent, in any combination.

The enhanced immune response may be an active or a passive immune response. Alternatively, the response may be part of an adoptive immunotherapy approach in which immune system cells, such as dendritic cells, B cells or macrophages, are obtained from an animal (e.g., a patient), then pulsed with composition comprising an antigenic composition. In this embodiment, the antigenic composition may comprise an additional immunostimulatory agent or a nucleic acid encoding such an agent. In a preferred embodiment, the animal (e.g., a patient) has or is suspected of having a cancer, and the cancer may be metastatic. In other embodiments the method of enhancing the immune response is practiced in conjunction with a cancer therapy, such as for example, a cancer vaccine therapy.

In one embodiment, an antigen presenting cell is utilized in the present invention. In general, the term “antigen presenting cell” can be any cell that accomplishes the goal of the invention by aiding the enhancement of an immune response (i.e., from dendritic cells, macrophage, monocytes and/or B-cells) against an antigen (e.g., a tumor antigen) or antigenic composition of the present invention. Such cells can be defined by those of skill in the art, using methods disclosed herein and in the art. As is understood by one of ordinary skill in the art (see for example Kuby, 1993, incorporated herein by reference), and used herein certain embodiments, a cell that displays or presents an antigen normally or preferentially with a class II major histocompatability molecule or complex to an immune cell is an “antigen presenting cell.” In certain aspects, a cell (e.g., an APC cell) may be fused with another cell, such as a recombinant cell or a tumor cell that expresses the desired antigen. Methods for preparing a fusion of two or more cells is well known in the art, such as for example, the methods disclosed in Goding, pp. 65-66, 71-74 1986; Campbell, pp. 75-83, 1984; Kohler and Milstein, 1975; Kohler and Milstein, 1976, Gefter et al., 1977, each incorporated herein by reference. In some cases, the immune cell to which an antigen presenting cell displays or presents an antigen to is a CD4+ TH cell. Additional molecules expressed on the APC or other immune cells may aid or improve the enhancement of an immune response. Secreted or soluble molecules, such as for example, cytokines and adjuvants, may also aid or enhance the immune response against an antigen. Such molecules are well known to one of skill in the art, and various examples are described herein.

Combination Treatments

In order to increase the effectiveness of the therapeutic compositions of the present invention, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

In a specific embodiment, the second agent for cancer treatment is a tumor antigen other than a specific mutated fibronectin, and in some embodiments it is a tumor antigen from a different mutation in fibronectin.

Gene Therapy

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that the compositions could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with the methods and/or compositions of the present invention. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

Biological Functional Equivalents

The present invention comprises methods and compositions concerning peptides. As modifications and/or changes may be made in the structure of the peptides of the present invention, it is within the scope of the present invention to include biologically functional equivalent molecules having similar or improved characteristics. A skilled artisan recognizes that a biologically functional equivalent of a tumor antigen is one in which it is still capable of eliciting an immune response against at least one tumor cell.. A skilled artisan also recognizes that a biologically functional equivalent of a tumor antigen peptide is one in which it is capable of enhancing an immune response when presented by the immune effector cell. In a specific embodiment, this includes providing protection against a subsequent challenge of the disease or pathogen.

Modified Peptides

Certain amino acids may be substituted for other amino acids in a peptide without appreciable loss of interactive binding capacity with structures such as, for example, binding sites on substrate molecules, receptors, and such like. So-called “conservative” changes do not disrupt the biological activity of the protein, as the structural change is not one that impinges on the peptide's ability to carry out its designed function. It is thus contemplated by the inventors that various changes may be made in the sequence of peptides disclosed herein, while still fulfilling the goals of the present invention.

In terms of functional equivalents, it is well understood by the skilled artisan that, inherent in the definition of a “biologically functional equivalent” protein and/or polynucleotide, is the concept that there is a limit to the number of changes that may be made within a defined portion of the molecule while retaining a molecule with an acceptable level of equivalent biological activity.

Amino acid substitutions are generally based on the relative similarity of the amino acid side-chain substituents, for example, their hydrophobicity, hydrophilicity, charge, size, and/or the like. An analysis of the size, shape and/or type of the amino acid side-chain substituents reveals that arginine, lysine and/or histidine are all positively charged residues; that alanine, glycine and/or serine are all a similar size; and/or that phenylalanine, tryptophan and/or tyrosine all have a generally similar shape. Therefore, based upon these considerations, arginine, lysine and/or histidine; alanine, glycine and/or serine; and/or phenylalanine, tryptophan and/or tyrosine; are defined herein as biologically functional equivalents.

To effect more quantitative changes, the hydropathic index of amino acids may be considered. Each amino acid has been assigned a hydropathic index on the basis of their hydrophobicity and/or charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (-1.6); histidine (−3.2); glutamate (−3.5); glutamine (−3.5); aspartate (−3.5); asparagine (−3.5); lysine (-3.9); and/or arginine (−4.5).

The importance of the hydropathic amino acid index in conferring interactive biological function on a protein is generally understood in the art (Kyte & Doolittle, 1982, incorporated herein by reference). It is known that certain amino acids may be substituted for other amino acids having a similar hydropathic index and/or score and/or still retain a similar biological activity. In making changes based upon the hydropathic index, the substitution of amino acids whose hydropathic indices are within ±2 is preferred, those which are within ±1 are particularly preferred, and/or those within ±0.5 are even more particularly preferred.

It also is understood in the art that the substitution of like amino acids can be made effectively on the basis of hydrophilicity, particularly where the biological functional equivalent protein and/or peptide thereby created is intended for use in immunological embodiments, as in certain embodiments of the present invention. U.S. Pat. No. 4,554,101, incorporated herein by reference, states that the greatest local average hydrophilicity of a protein, as governed by the hydrophilicity of its adjacent amino acids, correlates with its immunogenicity and/or antigenicity, i.e., with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those which are within ±1 are particularly preferred, and/or those within ±0.5 are even more particularly preferred.

Altered Amino Acids

The present invention, in many aspects, relies on peptides. These peptides may include the twenty “natural” amino acids, and post-translational modifications thereof. However, in vitro peptide synthesis permits the use of modified and/or unusual amino acids. A table of exemplary, but not limiting, modified and/or unusual amino acids is provided herein below.

TABLE 6
Modified and/or Unusual Amino Acids

| Abbr. | Amino Acid | Abbr. | Amino Acid |
|---|---|---|---|
| Aad | 2-Aminoadipic acid | EtAsn | N-Ethylasparagine |
| BAad | 3-Aminoadipic acid | Hyl | Hydroxylysine |
| BAla | beta-alanine, | AHyl | allo-Hydroxylysine |
| | beta-Amino-propionic acid | | |
| Abu | 2-Aminobutyric acid | 3Hyp | 3-Hydroxyproline |
| 4Abu | 4-Aminobutyric acid, | 4Hyp | 4-Hydroxyproline |
| | piperidinic acid | | |
| Acp | 6-Aminocaproic acid | Ide | Isodesmosine |
| Ahe | 2-Aminoheptanoic acid | Aile | allo-Isoleucine |
| Aib | 2-Aminoisobutyric acid | MeGly | N-Methylglycine, |
| | | | sarcosine |
| BAib | 3-Aminoisobutyric acid | MeIle | N-Methylisoleucine |
| Apm | 2-Aminopimelic acid | MeLys | 6-N-Methyllysine |
| Dbu | 2,4-Diaminobutyric acid | MeVal | N-Methylvaline |
| Des | Desmosine | Nva | Norvaline |
| Dpm | 2,2′-Diaminopimelic acid | Nle | Norleucine |
| Dpr | 2,3-Diaminopropionic acid | Orn | Ornithine |
| EtGly | N-Ethylglycine | | |

Mimetics

In addition to the biological functional equivalents discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Pharmaceutical Preparations

Pharmaceutical compositions of the present invention comprise an effective amount of one or more forms of a tumor antigen recognized by CD4+ T cells, such as a mutated fibronectin, in a pharmaceutically acceptable carrier or excipient. In a specific embodiment, the mutated fibronectin is a polynucleotide that comprises an A substitution for G at position 6427 and/or is a polypeptide that comprises a Glu to Lys substitution at amino acid position 2053. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one tumor antigen or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated. The carrier may comprise an adjuvant.

The targeting fusion form may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The targeting fusion form may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the mutant fibronectin is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

Site-Directed Mutagenesis

Structure-guided site-specific mutagenesis represents a powerful tool for the dissection and engineering of protein-ligand interactions (Wells, 1996, Braisted et al., 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase 1 (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et al., 1996; Burton and Barbas, 1994; Yelton et al., 1995; Jackson et al., 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. No. 5,798,208 and U.S. Pat. No. 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example1

### Exemplary Materials and Methods

Chemicals and Reagents

The following chemicals and reagents were purchased from the sources indicated: RPMI 1640, AIM-V medium, Lipofectamine, and G418 (GIBCO BRL, Gaithersberg, Md.); the eukaryotic expression vector pcDNA3.1 (Invitrogen, San Diego, Calif.); anti-HLA-DR2 monoclonal antibody (One lambda, Canoga Park, Calif.); and anti-immunoglobulin M antibody conjugated with fluorescent isothiocyanate (Vector Laboratories, Inc., Burlingame, Calif.).

Cell Lines and Cultures

CD4+ tumor-infiltrating lymphocytes (F27TIL) were cultured from a subcutaneous metastasis obtained from a melanoma patient by fine-needle aspiration (FNA). T cell clones or lines were grown in AIM-V medium containing 10% human AB serum and recombinant interleukin-2 (IL-2) (6000 WU/ml; Chiron, Emeryville, Calif.). Melanoma cell lines and Epstein-Barr virus (EBV)-transformed B cell lines were maintained in RPMI 1640 with 10% fetal calf serum (FCS). 293IMDR2 cells were grown in DMEM containing 10% FCS. The T cell clones were generated by limiting dilution methods (at 1 cell/well) from the CD4+ F27TIL cell line, and feeder allogeneic peripheral blood mononuclear cells (PBMCs) in RPMI1640 containing 10% human AB sera and 300 IU IL-2. To obtain optimal expansion, the inventor used the OKT3 expansion method as previously described (Wang et al., 1996; Wang et al., 1998). Melanoma cell lines and EBV-transformed B-cell lines used in this study were cultured in RPMI 1640 medium containing 10% FCS. 293IMDR2 cells was established by transfecting plasmid DNA encoding DRB1*1501 CDNA into 293IMA cells expressing the Ii, DMA, DMB, and DRA genes, and were selected with RPMI 1640/10% FCS containing blasticidin S (25 μg/ml). HLA-DR2-positive cells were sorted by FACS using DR2-specific antibodies.

cDNA Library Construction

Total RNA was extracted from F27 mel cells using Trizol reagent (GIBCO BRL, Gaithersburg, Md.). Poly(A) RNA was purified from total RNA by the polyATract system (Promega, Madison, Wis.) and converted to cDNA using a cDNA construction kit (GIBCO BRL) with an oligo-dT primer. The cDNA inserts were then ligated to a pTSX vector containing an Ii fragment (amino acid 1-80) (Wang et al., 1999), and cDNA libraries were electroporated into DH10B cells (GIBCO BRL). Plasmid DNA for cDNA library pools was prepared from bacteria, each consisting of approximately 100 CDNA clones.

cDNA library Screening and GM-CSF Secretion Assay

DNA transfection and GM-CSF assays were performed as previously described (Wang et al., 1999a; Wang et al., 1999b). Briefly, 200 ng of cDNA pools were mixed with 2 μl of Lipofectamine in 100 μl of serum-free DMEM for 15-45 min. The DNA/Lipofectamine mixture was then added to the 293IMDR2 cells (5×104) and incubated overnight. The following day, cells were washed twice with AIM-V medium. CD4+ T cells were then added at a concentration of 5×10⁴ cells/well in AIM-V medium containing 120 lU/ml of IL-2. After an 18-24 h of incubation, 100 μl of supernatant was collected and GM-CSF concentrations were measured in a standard ELISA assay (R+D Systems, Minneapolis, Minn.). For testing peptide recognition, 888EBV were incubated with peptides at 37° C. for 90 min, and then washed three times with AIM-V medium containing 120 IU/ml of IL-2. T cells were added and incubated for an additional 18-24 h.

Northern Blot Analysis

Total RNA from human normal tissue was purchased from Clontech. (Palo Alto, Calif.). Twenty jig of total RNA was subjected to electrophoresis in a 1.2% formaldehyde agarose gel and transferred to a nylon membrane. DNA fragments of the FN gene were labeled with [α-³²P]-dCTP by the random priming method. Prehybridization and hybridization were performed according to the QuickHyb protocol (Stratagene, La Jolla, Calif.). Membranes were washed twice with 2× SSC/0.1% SDS at room temperature for 15 min and twice with 0.1× SSC/0.1% SDS at 60° C. for 30 min. Autoradiography was performed at −70° C. An actin probe was used to serve the internal control for the amount of total RNA loading onto the gel.

Peptides Synthesis and T-cell Epitopes

The peptides were synthesized by a solid-phase method using a peptide synthesizer (Model AMS 422, Gilson Co., Inc., Worthington, Ohio). Some peptides were purified by HPLC and had greater than 98% purity. The mass of some peptides was confirmed by mass spectrometry analysis. Peptides reactive with CD4+ F27TIL-T1 cells were identified and characterized as previously described (Wang et al., 1999; Wang et al., 1999).

Cloning of Full-Length Fibronectin cDNA

A SuperScript II RT kit (Invitrogen, Inc. San Diego, Calif.) was used in reverse transcription. 20 μl reverse transcription mixture contained 2 μg of total RNA and was incubated at 42° C. for 1 h. After reverse transcription, 2.5 μl reverse transcription mixture was used in PCR. 50 μl of PCR reaction mixture contained 5 μl of 10× ThermalAce buffer, 1 μl of 50× dNTP mixture, 200 μM of primer FN5P2 (5′ CTCAACATGGTTAGGGGTCCGGGGCCCGGGCTG; SEQ ID NO:5), 200 μM of primer FN3P1 (5′ AGAGACATGCTTGTTCCTCTGGA 3′; SEQ ID NO:6) and 2 μl ThermalAce DNA polymerase (Invitrogen, Inc. San Diego, Calif.). Primer FN5P2 is a sense primer, corresponding to the 5′-end sequence of fibronectin gene. Primer FN3P1 is an antisense primer, corresponding to the 3′-end sequence of the fibronectin gene. The PCR amplification program included an initial step at 95° C. for 2 min, 30 cycles of 30 sec at 95° C., 30 sec at 65° C., and 10 min at 72° C., and a final step at 72° C. for 15 min. Then, 50 μl of PCR product was supplemented with 1 μl of Taq DNA polymerase (Invitrogen, Inc. San Diego, Calif.), and incubated 72° C. for 30 min. 50 μl PCR product was separated on a 0.8% (WNV) agarose gel. The desired bands of PCR product were gel-purified, and then cloned into pCR-XL-TOPO® vector (Invitrogen, Inc. San Diego, Calif.). Individual Plasmid DNAs were prepared and sequenced to confirm the correctness of full-length wild type and point-mutated FN cDNAs.

Transfection of Fibronectin Genes into Tumor Cells

Both wild-type and mutated FN gene fragments were released with Hind III and Not I from the pCR-XL-TOPO vector, and subcloned into a pcDNA3.1/zeocin vector (Invitrogen, Inc. San Diego, Calif.). Plasmid DNAs encoding either wild-type or mutated FN cDNA, as well as a pcDNA3.1/zeocin empty vector were transfected into 1143 mel tumor cells in 6-well plates with LipofectAMINE (GIBCO BRL). Transfected tumor cells were selected in the RPMT1640/10% FCS containing 250 μg/ml Zeocin (Invitrogen, Inc. San Diego, Calif.).

Immunostaining of Extracellular Matrix with Anti-fibronectin Antibody

1143 mel, 1195 mel, F27 mel tumor cells and FN-transfected tumor cells were cultured overnight in 6-well plates containing a coverslip. Cells attached to the coverslips were fixed in PBS/4% formaldehyde at room temperature for 20 min, and washed three times with phosphate-buffered saline (PBS). The coverslips were kept in acetone at −20° C. for 3 min, dried in the air, and washed three times with PBS. They were then blocked in PBS/5% FCS in a moist chamber at 37° C. for 20 min, washed with PBS for 5 min, and incubated with 1:500 anti-fibronectin monoclonal antibody (Ab-9, NeoMarkers, Fremont, Calif.) in a moist chamber at 37° C. for 1 h. Similar experiments with other anti-fibronectin antibodies Ab-1, Ab-7, Ab-8 and Ab-9 (NeoMarkers, Fremont, Calif.) were also performed. The slides were washed three times with PBS, blocked with PBS/5% FCS at room temperature for 20 min, washed two times with 1× PBS, incubated with 1:20 Texas Red-conjugated anti-mouse IgG in the dark in a moist chamber at 37° C. for 30 min, and washed five times with PBS. They were kept in PBS overnight at 4° C., mounted in PBS/50% glycerol, and viewed with a fluorescence microscope and photographed at 40× magnification.

Migration Assays

2.5×10⁴ 1143 mel tumor cells and transfected tumor cells were seeded into the upper chamber with 0.75 ml of RPMI-1640 serum-free medium in BIOCOAT MATRIGEL Cells (Becton Dickinson, San Jose, Calif.). The bottom chamber contained 0.75 ml of RPMI-1640 serum-free medium (control medium) or 0.75 ml of RPMI-1640/10% FCS (complete medium). After incubation at 37° C. and 5% CO₂ for 48 h, Cells in the lower chambers were fixed in 4% formaldehyde in PBS at room temperature for 20 min, stained with crystal violet, and examined and counted from four different fields under a compound microscope.

Immunoprecipitation and Western Blotting

1143 mel, 1195 mel and F27 mel were lysed in lysis buffer on ice. After spinning, the cell lysates were immunoprecipitated with an anti-β1 integrin antibody and pulled down with the protein A-beads. The immunoprecipitated complexes were separated on SDS-PAGE. After membrane transfer, proteins were detected with anti-FN or anti-β1 integrin antibodies. For the detection of FN in whole cell lysates, 1143 mel tumor cells, 1195 mel and F27 mel tumor cells from T75 flasks with 80-90% confluence were trypsinized. After spinning, the cell pellets were collected and redissolved in 1× SDS loading buffer (50 mM Tris-HCl, pH6.8, 100 mM DTT, 2% SDS, and 10% glycerol). The protein concentration was determined with Bio-Rad Protein Assay Dye Reagent (Bio-Rad, Hercules, Calif.). Fifteen pg of total protein for each sample was loaded onto an 8% SDS-polyacrylamide gel, separated at 100 V constant for 1 h, and transblotted onto a nitrocellulose membrane (BA83, Schleicher & Schuell, Keene, N.H.) at 100 V constant for 1 h. The membrane was blocked with 3% milk for 1 h, incubated with 1:200 anti-FN mAb for 1 h, and washed three times with TBST (10 mM Tris-HCl, pH 8.0, 150 mM NaCl, and 0.05% Tween-20) for 5 min. The membrane was incubated with 1:3000 goat anti-mouse horseradish peroxidase (HRP)-conjugated IgG for 1 h, washed three times with TBST for 5 min, rinsed with water, detected with LumiGLO chemiluminescent substrate (Kirkegaar & Perry Laboratories, Gaithersburg, Md.), exposed to X-OMAT AR film (Eastman Kodak, Rochester, N.Y.), developed automatically, and photographed.

### Example2

### Recognition of Autologous Tumor Cells by HLA-DR2-Restricted CD4+ T Cells

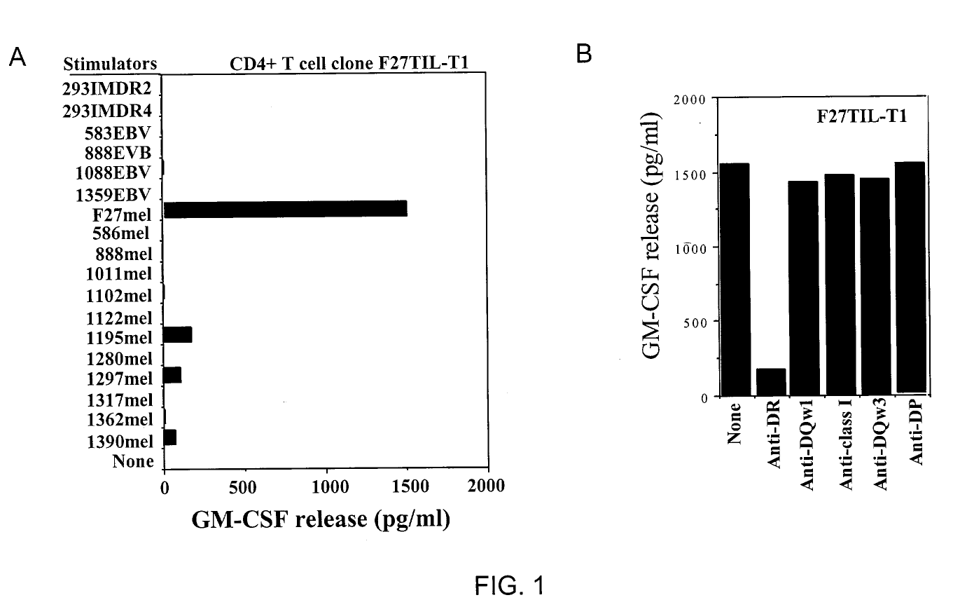

In a search for tumor-reactive CD4+ T cells, the inventor recently established four CD4+ T cell clones from F27 TIL cells. He recognized the autologous tumor cell line F27 mel, but did not respond to autologous PBMC, MHC class II-matched or -mismatched tumor cell lines, or 293 cells expressing DR2 molecules. Representative data from one such clone (F27TIL-T1) are shown in FIG. 1A. To determine the restriction elements used by T cells, the inventor tested T-cell recognition in the presence or absence of specific antibodies. As shown in FIG. 1B, T-cell reactivity against F27 mel cells was specifically blocked by a monoclonal antibody (mAb) against HLA-DR, but not by mAb against HLA-DQ, HLA-DP or MHC class I molecules. HLA typing analysis of F27 mel cells indicated that only HLA-DR2 was expressed in tumor cells, suggesting that CD4+ F27TIL-T1 and other clones recognized a mutated or unique tumor antigen presented by HLA-DR2. For this reason, HLA-DR2 was selected as the restriction element for the initial cDNA library screening.

### Example3

### Identification of Mutated Fibronectin as a Tumor Antigen Recognized by CD4+ T Cells

To isolate the gene encoding a tumor antigen recognized by CD4+ F27TIL-T1, the inventor used a genetic targeting expression (GTE) system that has been successfully employed to identify several MHC class II-restricted tumor antigens (Wang et al., 1999). This system uses a 293-based cell line as professional antigen presenting cells (APC) and an li-fused cDNA library. To generate 293IMDR2 cells expressing Ii, DMA, DMB, DRA and DRB1*1501, the inventor first cloned HLA-DRB1*1501 cDNA from the autologous F27 mel cells into a pEF6NV5-His/TOPO expression vector containing a blasticidin S resistance gene. The cDNA sequence was confirmed to be that of DRB1*1501. 293 cells stably expressing the other four genes (Ii, DMA, DMB and DRA), but not DRB1*1501, were then transfected with the DR2 expressing plasmid DNA. After selection with blasticidin S for 2 weeks, the 293IMDR2 cell line was screened by FACS analysis using a DR2-specific antibody, and further expanded for use as APC for library screening.

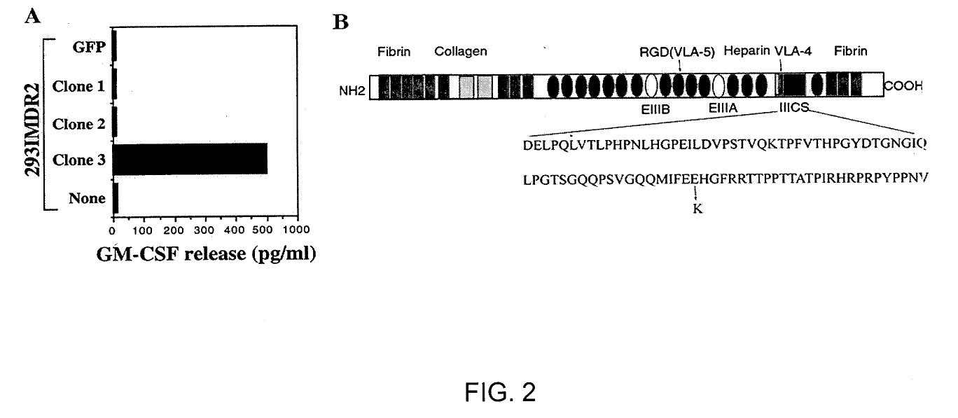

The inventors next constructed a cDNA library by fusing a targeting sequence of invariant chain to the 5′ end of cDNAs derived from F27 mel tumor cells. As previously demonstrated, this strategy will target Ii-fusion proteins translated from the Ti-fusion library to the endosomal/lysosomal compartment for efficient antigen processing and presentation to T cells (Wang et al., 1999). cDNA subpools with approximately 100 cDNA clones per pool were prepared. The F27 mel cDNA library was then introduced into 293IMDR2 expressing DMA, DMB, Ii, DRA and DRB1*1501 molecules. After screening a total of2×10⁵ cDNA clones, the inventor identified five positive cDNA pools that conferred T-cell recognition by CD4+ F27TIL-T1 cells when transfected into 293IMDR2 cells. The individual positive clones were then isolated from the positive cDNA pools and tested for recognition by CD4+ F27TIL-T1 cells (FIG. 2A). Evaluation of three other CD4+ T cell clones showed that they were capable of recognizing the same positive cDNA clone isolated from the F27 cDNA library with F27TIL-T1 cells.

DNA sequencing analysis revealed that cDNA clone 3 contained a DNA fragment encoding the FN protein involved in extracellular ECM formation, tumor transformation and metastasis (FIG. 2B). All DNA sequences from five different cDNA pools were identical to the published sequence with the exception of one nucleotide (A) substitution for G at position 6427, based on the numbering of the published FN sequence (Accession number NM-002026) (16). This point mutation (G to A) at amino acid position 2053 was found in all positive cDNA clones, resulting in a nonconservative substitution of Lys (positively charged residue) for Glu (negatively charged residue) (FIG. 2B). This mutation was located in the region of type III repeats (IIICS) of cellular FN that is involved in the formation of ECM formation (Gutman and Komblihtt, 1987). In addition, DNA sequences from the amplified DNA fragments from autologous T cells or PBMC were identical to the published wild type sequence, suggesting that this is a somatic mutation occurred in cancer cells.

### Example4

### Mutated Peptides Recognized by CD4+ T Cells

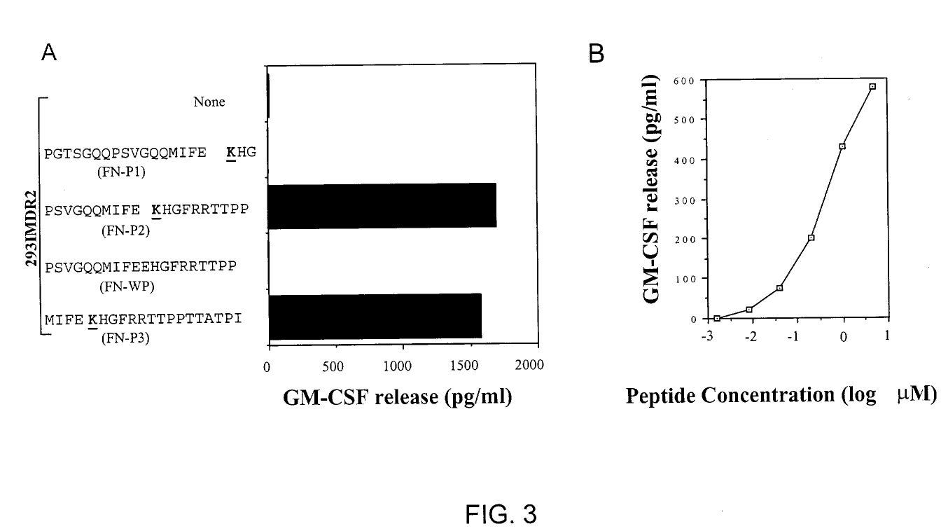

To confirm if T cell recognition was restricted by HLA-DR2, the inventor transfected cDNA clone 3 into both 293IMDR2 and 293IMDR4 cells, and then tested for recognition by CD4+ F27TIL-T1 cells. CD4+ T cells recognized 293DR2 cells transfected with the mutant FN cDNA, but not 293DR4 cells transfected with the wild-type FN cDNA or 293DR4 cells transfected with the mutant FN cDNA, suggesting that T-cell epitopes are located in the region with a point mutation. To identify the antigenic epitopes recognized by CD4+ T cells, a wild-type peptide and three overlapping 20-mer peptides containing the mutated residue were generated, and their ability to activate T cells was tested. Two of three mutant peptides were recognized by CD4+ F27TIL-T1 (FIG. 3A). These two mutated peptides shared 13 amino acids (MIFEKHGFRRTTPP; SEQ ID NO: 1), suggesting that this peptide sequence contained a T-cell epitope for T-cell recognition. By contrast, T cells failed to recognize the wild type peptide, which lacked the mutant residue, but otherwise was identical to the mutated peptides. Peptide titration experiments showed that T cell recognition could be readily detected at a peptide concentration of approximately 500 nM (FIG. 3B).

### Example5

### Mutant FN Disrupts FN Matrix Formation and Enhances the Metastatic Potential of Tumor Cells

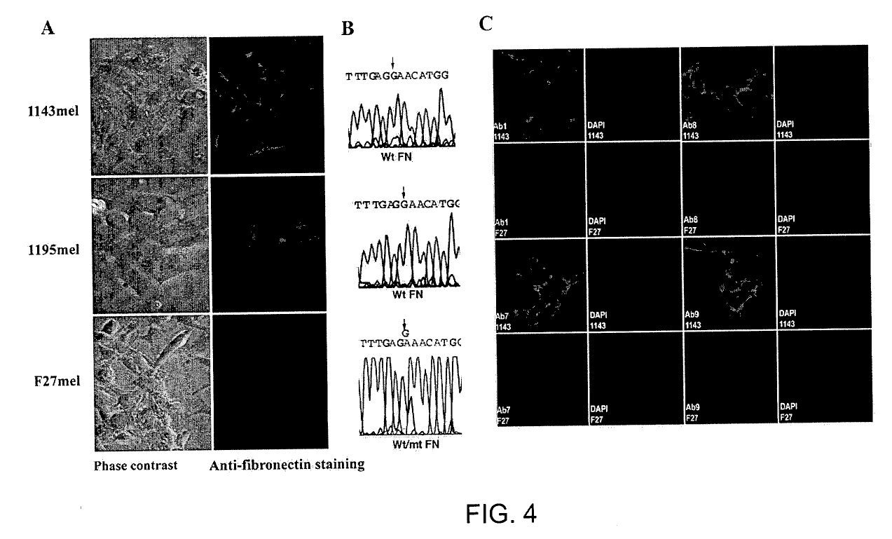

Since tumor cells are a heterogeneous population, in some embodiments some cells in the primary tumor cells might not contain the FN mutation, while this G-to-A substitution should be found in all metastatic lesions, provided this mutation affects the metastatic potential of tumor cells. To test this embodiment, 53 tumor cell clones were generated by the limiting dilution method from fresh tumor cells derived from different metastatic lesions. After PCR amplification and sequencing of genomic DNA, the presence of the mutant FN gene in all tumor cell clones was identified, strongly suggesting that the mutation in FN plays a role in subsequent tumor metastasis. By contrast, two control tumor cell lines, 1143 mel and 1195 mel, contained the wild-type FN allele exclusively. Representative data are shown in FIG. 4B.

Because FN plays a significant role in FN matrix formation (Akamatsu et al., 1996; Roman et al., 1989), the inventor examined FN matrix formation in F27 mel as well as other tumor cell lines (1143 mel and 1195 mel) by staining with an anti-FN Ab-9 antibody. As shown in FIG. 4A, intense FN staining was evident in 1143 mel and 1195 mel, but was significantly reduced in F27 mel cells. In contrast to 1143 mel and 1195 mel cell lines, which contained wild type FN only, F27 mel cells contained both wild type and mutant FN (FIG. 4B). Thus, the point mutation in FN results in the loss of ability to form FN matrices. The present inventor also stained for extracellular FN in tumor cell lines with 3 different anti-FN antibodies (Ab-1, Ab-7 and Ab-8) that recognize epitopes located in different regions of the FN molecule. The resultant staining patterns were either identical or similar to that obtained with Ab-9 (FIG. 4C), indicating that the point mutation in FN did not alter its recognition by anti-FN antibodies.

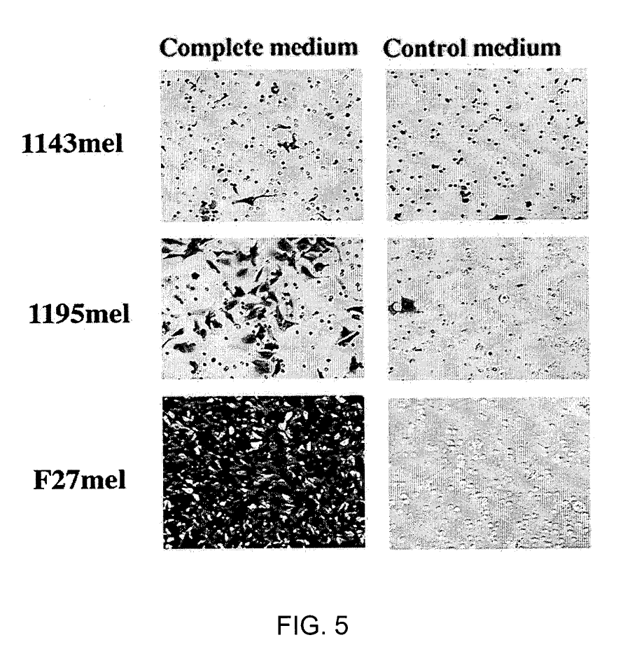

The loss of FN matrix formation has been implicated in tumor transformation (Giancotti and Ruoslahti, 1990; Akamatsu et al., 1996). Tumor cells with metastatic potential must migrate away from the primary tumor, invade and implant in a distant site for re-establishment of new tumor. Thus, modulation of FN matrix formation is a critical step in tumor metastasis (Clark et al., 2000). To characterize that the loss or reduction of FN matrix formation in F27 mel cells enhances tumor metastasis migration assays were performed to measure the ability of F27 mel, 1143 mel and 1195 mel tumor cells to migrate through matrigel from one chamber containing control medium to another containing complete growth medium. Interestingly, relatively few of the 1143 mel and 1195 mel tumor cells with the wild-type FN gene and normal FN matrix formation were capable of migrating to the well containing the complete medium. In contrast, the F27 mel tumor cells readily migrated to the well with complete medium in more than 20 fold higher numbers than observed with that 1143 mel and 1195 mel (FIG. 5). These results demonstrate that the FN mutation contributes to subsequent loss of FN matrix formation, leading to the enhanced metastatic potential of tumor cells.

### Example6

### Dominant Negative Effect on FN Matrix Formation

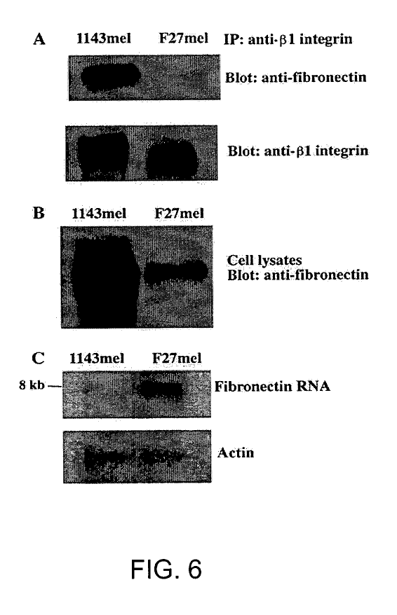

Since interaction of FN with its receptor, an integrin heterodimers (α5β1) is important to ECM formation (Giancotti and Ruoslahti, 1990), it was next tested whether mutant FN may alter protein interaction between FN and integrin. FN-integrin complexes were first immunoprecipitated with anti-β1 integrin Ab. As shown in FIG. 6A, similar amounts of integrin was pulled down by anti-β1 integrin in the lysates of 1143 mel and F27 mel cells, but the amount of FN that formed complexes with integrins in F27 mel was much less than that in 1143 mel tumor cells (FIG. 6A). Because the antibody used could not distinguish wild type from mutant FN proteins, the total amount of FN in 1143 mel and F27 mel lysates was also checked, and found that the total amount of FN (both wild-type and mutant) in F27 mel was much lower than that in 1143 mel (FIG. 6B). Thus, the overall difference in the total amount of FN in the cell lysates of 1143 mel and F27 mel may account for the difference in amount of FN in the FN-integrin complexes immunoprecipitated from 1143 mel and F27 mel.

To exclude the possibility that the low level of FN in F27 mel cells is due to low expression at mRNA level, Northern blot analysis was performed. Interestingly, mRNA level of FN in F27 mel was much higher than that in 1143 mel cells, while actin probe hybridization showed the equal amount of RNA loading (FIG. 6C), suggesting that the mutation in FN, rather than RNA expression level, reduces the amount of FN and its complexes in F27 mel cells. In other words, mutant FN has a dominant negative effect on FN matrix formation, in some embodiments.

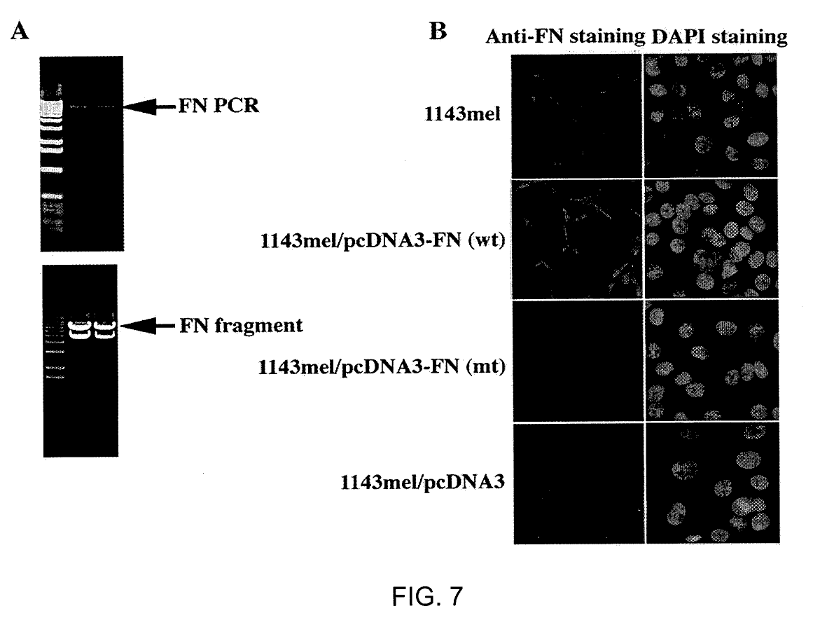

To determine whether the point mutation in FN is responsible for the loss of FN antibody staining in FN matrices and the enhanced migration of tumor cells, wild-type and mutant FN cDNAs were cloned from F27 mel in a pcDNA3.1Z expression vector (FIG. 7A). The sequences of both wild-type and mutant cDNA clones were confirmed by DNA sequencing analysis. These clones were full-length cDNAs containing ED-A, ED-B and IIICS segments (FIG. 2B), and the mutation (G-to-A) at amino acid position 2053 was found in the mutated cDNA, but otherwise was identical to the wild-type cDNA. With these constructs, it was further characterized whether the point mutation in FN was responsible for the observed phenotypic changes. 1143 mel was transfected with pcDNA3-FN (wt) or pcDNA3-FN (mt) and selected in culture medium containing Zeocin (250 μg/ml). Twelve clones expressing the wild-type FN construct were established for 1143 mel, 22 clones expressing mutant FN in 1143 mel, and 9 clones for empty vector (control cell line). The presence and expression of the mutant FN gene were confirmed by RT-PCR and sequencing analysis. FN staining was performed to assess the effect of the transfected FN gene on matrix formation and cell migration. Representative data from three independent studies with different 1143 mel cell clones stably expressing wild type FN, mutant FN or empty vector are shown in FIG. 7B. The similar staining patterns of FN matrices were found for all three independent clones examined for each construct. The parental cell line 1143 mel and 1143 mel/pcDNA (empty vector) exhibited a comparable staining intensity of FN matrices, while 1143 mel/pcDNA3-FN (wt) cells resulted in more intense staining. By contrast, 1143 mel cells stably expressing pcDNA3-FN(mt) showed little or no staining of FN matrices compared with other cell lines/clones containing wild-type FN, demonstrating that expression of mutant FN in 1143 mel cells abolishes immunostaining for endogenous wild-type FN and/or ectopic mutant FN in the matrix assembly of tumor cells.

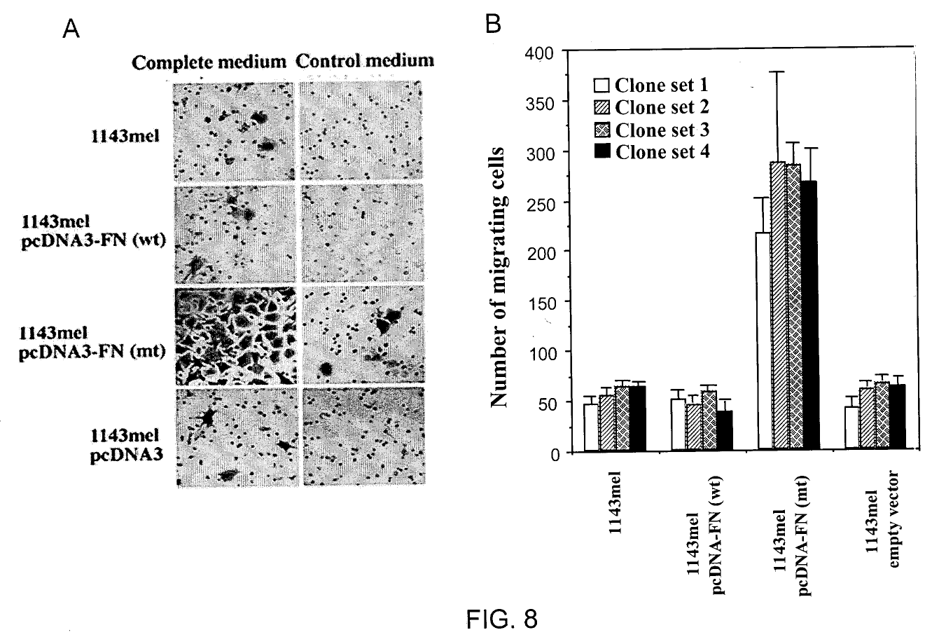

The migratory ability of these cell lines expressing wild type or mutant FN was further evaluated. As shown in FIG. 8A, 1143 mel, 1143 mel/pcDNA3-FN (wt) and 1143 mel/pcDNA3 cells displayed little or no migration in a matrigel assay. However, 1143 mel/pcDNA3-FN (mt) cells greatly gained the ability to migrate from one chamber containing control medium to another containing growth medium, indicating that the mutant FN enhances the metastatic potential of tumor cells. To rule out clonal variation, similar experiments were repeated with four sets of independent tumor cell clones expressing either wild-type, mutant FN or empty vector. FIG. 8B, a summary of results obtained from these experiments, shows that all tumor cell clones expressing mutant FN had an increased ability to migrate compared with cell clones expressing wild-type FN or an empty vector. The number of migrating cells in 1143 mel/pcDNA-FN (mt) clones was at least four-fold higher than that of other groups that express either wild-type FN or empty vector. The P-value for significant difference is 0.0007 among the two comparison groups of cell clones expressing mutant FN and other cell clones expressing either wild-type FN, empty vector. There were no statistically significant differences among groups of parental 1143 mel and its derivative clones expressing wild-type or empty vector. These results indicate that expression of mutant FN greatly enhance the migratory capacity of tumor cells.

### Example7

### Significance of the Results

Because of the importance of CD4+ T cells in antitumor immunity, much effort has been directed toward identifying MHC class II-restricted tumor antigens. Thus far, only DR1- or DR4-restricted tumor antigens have been identified with tumor-reactive CD4+ T cells established from tumor infiltrating lymphocytes (Houghton et al., 2001; Wang, 2001). Due to the polymorphic properties of MHC class II molecules, DR1 or DR4 expression accounts for 15-20% of the population (Zeng et al., 2001). Identification of tumor antigens presented by other relatively dominant alleles such as DR2, DR3 or R7 (each accounting for 20-25% of the population) remains critically important for the development of effective vaccines by recruiting both CD4+ and CD8+ T cells. Indeed, T cell recognition of potentially shared tumor antigens presented by DR2 (DRP1*1501) molecules has been reported (Takahashi et al., 1995), but the identity of such antigens remains unknown.

As shown herein, the inventor identified a novel mutated form of FN as a tumor antigen recognized by CD4+ F27TIL using a genetic targeting expression approach. The findings presented strongly suggest that tumor-reactive CD4+ T cells plays a significant role in eliminating metastatic cancer cells. FN is a gene product critical for extracellular matrix formation and indispensable for vertebrate embryogenesis (George et al., 1993). FN forms a complex with its receptor integrins (α5β1) for the initiation of ECM formation. Loss of capacity to form an FN containing ECM has been suggested as a feature of the transformed phenotype of cancer cells, and restoration of ECM formation correlates with reduced malignancy of cancer cells (Hynes et al., 1978; Giancottie and Ruoslahti, 1990; Akamatsu et al., 1996). Recently, several groups using microarray technology demonstrated a link between FN expression at the RNA level and tumor metastasis (Clark et al., 2000; Maniotis et al., 1999; MacDonald et al., 2001). Interestingly, the Northern blot data are consistent with these published results, which show that FN mRNA is approximately four-fold higher in metastatic versus poorly metastatic tumor cells. However, the protein level of FN in highly metastatic tumor cells (F27 mel) was much lower than in poorly metastatic tumor cells (1143 mel). The reduced FN protein in metastatic tumor cells was associated with significant reduction of FN matrix formation. Because introduction of the mutant FN into tumor cells expressing wild-type FN resulted in significant loss of FN matrix formation while the empty vector did not, the inventors believed that the point mutation, which resulted in substitution of Lys for Glu, was responsible for the observed phenotype. More importantly, the inventor demonstrated that this point mutation in FN converted poorly metastatic tumor cells to highly metastatic ones. This is the first report showing that mutated FN directly affects extracellular FN matrix formation, and thus enhances the metastatic potential of tumor cells.

The results indicate that CD4+ F27TIL-T1 cells recognized a mutated FN presented by DR2 molecules. Although several mutated forms of proteins such as CDC27 (nuclear protein) involved in cell cycle regulation, TPI (cytosolic protein) required for energy production, and the secretory fusion protein LDFP resulting from chromosomal rearrangement, have been identified as class II-restricted tumor antigens, they are presented by either DR1 or DR4 molecules. Of particular interest is that the majority of MHC class II-restricted tumor antigens identified by tumor-reactive CD4+ T cells derived from cancer patients are mutated or fusion proteins. By contrast, the majority of the MHC class I-restricted tumor antigens recognized by CD8+ CTLs are nonmutated self-antigens (Wang and Rosenberg, 1999). All of the known MHC class I-restricted human melanoma antigens, such as tyrosinase, gp 100, MAGE-3 and NY-ESO-1, also contained CD4+ T cell epitopes, but they were identified either by stimulation of human PBMC with DCs pulsed with peptides or proteins (Kobayashi et al., 1998; Chaux et al., 1999; Manici et al., 1999; Zarour et al., 2000; Jager et al., 2000) or by the use of HLA-DR4 transgenic mice (Zeng et al., 2000; Touloukian et al., 2000). None of them with the exception of tyrosinase were identified by tumor-reactive CD4+ T cells derived from patients, although the mechanism responsible for this is not understood. Since both animal and human studies indicate that CD4+ T cells play a central role in initiating and maintaining host immune responses against cancer, the presence of such self-reactive CD4+ T cells may cause self-tissue destruction. Self-antigens such as gp100 and tyrosinase specific CD4+ T cells could be induced following stimulation of PBMCs with peptides in vitro. However, such T cells exhibited a relatively low affinity for MHC-peptide complexes compared to T cells specific for mutated tumor antigens, and required a high concentration of peptide for T-cell recognition (Touloukian et al., 2000; Topalian et al., 1996).

While vaccines containing nonmutated shared class II-restricted tumor antigens may be useful in a broad coverage of cancer patients, mutated tumor antigens may have a limited clinical application. Nonetheless, several MHC class II-restricted mutated murine tumor antigens have been identified by a biochemical approach (Monach et al., 1995; Matsutake and Srivastava, 2001). Immunization with T helper peptides from viral or the mutated proteins can effectively reject MHC class II-negative tumor cells, suggesting the importance of CD4+ T cell response in antitumor immunity (Matsutake and Srivastava, 2001; Ossendorp et al., 1998; Toes et al., 1999; Mumberg et al., 1999). Moreover, in addition to providing help for CD8+ T cells (38), in some embodiments of the present invention CD4+ T cells play a far broader role in orchestrating the host response to tumor (Hung et al., 1998) and autoimmune diseases (Van de Keere and Tonegawa, 1998).

### References

All patents and publications mentioned in the specification are indicative of the level of those skilled in the art to which the invention pertains. All patents and publications are herein incorporated by reference to the same extent as if each individual publication was specifically and individually indicated to be incorporated by reference.

### Patents

U.S. Pat. No. 4,554,101

U.S. Pat. No. 5,446,128

U.S. Pat. No. 5,710,245

U.S. Pat. No. 5,840,833

U.S. Pat. No. 5,859,184

U.S. Pat. No. 6,475,488

U.S. patent application Ser. No. 10/077,555

### Publications

Akamatsu, H., K. Ichihara-Tanaka, K. Ozono, W. Kamiike, H. Matsuda, and K. Sekiguchi. 1996. Suppression of transformed phenotypes of human fibrosarcoma cells by overexpression of recombinant fibronectin. Cancer Res. 56:4541-4546.

Boon, T., J.-C. Cerottini, B. Van Den Eynde, P. Van der Bruggen, and A. Van Pel. 1994. Tumor antigens recognized by T lymphocytes. Annu. Rew. Immunol. 12:337-365.

Chaux, P., V. Vantomme, V. Stroobant, K. Thielemans, J. Corthals, R. Luiten, A. M. Eggermont, T. Boon, and P. van der Bruggen. 1999. Identification of MAGE-3 epitopes presented by HLA-DR molecules to CD4(+) T lymphocytes. J. Exp. Med. 189:767-778.

Clark, E. A., T. R. Golub, E. S. Lander, and R. O. Hynes. 2000. Genomic analysis of metastasis reveals an essential role for RhoC. Nature 406:532-535.

George, E. L., E. N. Georges-Labouesse, R. S. Patel-King, H. Rayburn, and R. O. Hynes; 1993. Defects in mesoderm, neural tube and vascular development in mouse embryos lacking fibronectin. Development 119:1079-1091.

Giancotti, F. G., and E. Ruoslahti. 1990. Elevated levels of the alpha 5 beta 1 fibronectin receptor suppress the transformed phenotype of Chinese hamster ovary cells. Cell 60:849-859.

Gutman, A., and A. R. Kornblihtt. 1987. Identification of a third region of cell-specific alternative splicing in human fibronectin mRNA. Proc. Natl. Acad. Sci. U.S.A. 84:7179-7182.

Houghton, A. N., J. S. Gold, and N. E. Blachere. 2001. Immunity against cancer: lessons learned from melanoma. Curr. Opin. Immunol. 13:134-140.

Hung, K., R. Hayashi, A. Lafond-Walker, C. Lowenstein, D. Pardoll, and H. Levitsky. 1998. The central role of CD4(+) T cells in the antitumor immune response. J. Exp. Med. 188:2357-2368.

Hynes, R. O., I. U. Ali, A. T. Destree, V. Mautner, M. E. Perkins, D. R. Senger, D. D. Wagner, and K. K. Smith. 1978. A large glycoprotein lost from the surfaces of transformed cells. Ann. N.Y. Acad. Sci. 312:317-342.

Jager, E., D. Jager, J. Karbach, Y. T. Chen, G. Ritter, Y. Nagata, S. Gnjatic, E. Stockert, M. Arand, L. J. Old, and A. Knuth. 2000. Identification of NY-ESO-1 epitopes presented by human histocompatibility antigen (HLA)-DRB4*0101-0103 and recognized by CD4(+) T lymphocytes of patients with NY-ESO-1-expressing melanoma. J. Exp. Med. 191:625-630.

Kalams, S. A., and B. D. Walker. 1998. The critical need for CD4 help in maintaining effective cytotoxic T lymphocyte responses. J. Exp. Med. 188:2199-2204.

Kobayashi, H., T. Kokubo, K. Sato, S. Kimura, K. Asano, H. Takahashi, H. Tizuka, N. Miyokawa, and M. Katagiri. 1998. CD4+ T cells from peripheral blood of a melanoma patient recognize peptides derived from nonmutated tyrosinase. Cancer Res. 58:296-301.

Komblihtt, A. R., K. Vibe-Pedersen, and F. E. Baralle. 1983. Isolation and characterization of cDNA clones for human and bovine fibronectins. Proc. Natl. Acad. Sci. U.S.A. 80:3218-3222.

MacDonald, T. J., K. M. Brown, B. LaFleur, K. Peterson, C. Lawlor, Y. Chen, R. J. Packer, P. Cogen, and D. A. Stephan. 2001. Expression profiling of medulloblastoma: PDGFRA and the RAS/MAPK pathway as therapeutic targets for metastatic disease. Nat. Genet. 29:143-152.

Mandruzzato, S., F. Brasseur, G. Andry, T. Boon, and P. van der Bruggen. 1997. A CASP-8 mutation recognized by cytolytic T lymphocytes on a human head and neck carcinoma. J. Exp. Med. 186:785-793.

Manici, S., T. Stumiolo, M. A. Imro, J. Hammer, F. Sinigaglia, C. Noppen, G. Spagnoli, B. Mazzi, M. Bellone, P. Dellabona, and M. P. Protti. 1999. Melanoma cells present a MAGE-3 epitope to CD4(+) cytotoxic T cells in association with histocompatibility leukocyte antigen DR11. J. Exp. Med. 189:871-876.

Maniotis, A. J., R. Folberg, A. Hess, E. A. Seftor, L. M. Gardner, J. Pe'er, J. M. Trent, P. S. Meltzer, and M. J. Hendrix. 1999. Vascular channel formation by human melanoma cells in vivo and in vitro: vasculogenic mimicry. Am. J. Pathol. 155:739-752.

Matsutake, T., and P. K. Srivastava. 2001. The immunoprotective MHC II epitope of a chemically induced tumor harbors a unique mutation in a ribosomal protein. Proc. Natl. Acad. Sci. U.S.A. 98:3992-3997.

Monach, P. A., S. C. Meredith, C. T. Siegel, and H. Schreiber. 1995. A unique tumor antigen produced by a single amino acid substitution Immunity 2:45-59.

Mumberg, D., P. A. Monach, S. Wanderling, M. Philip, A. Y. Toledano, R. D. Schreiber, and H. Schreiber. 1999. CD4(+) T cells eliminate MHC class II-negative cancer cells in vivo by indirect effects of IFN-gamma. Proc. Natl. Acad. Sci. U.S.A. 96:8633-8638.

Ossendorp, F., E. Mengede, M. Camps, R. Filius, and C. J. Melief. 1998. Specific T helper cell requirement for optimal induction of cytotoxic T lymphocytes against major histocompatibility complex class II negative tumors. J. Exp. Med. 187:693-702.

Pieper, R., R. E. Christian, M. I. Gonzales, M. I. Nishimura, G. Gupta, R. E. Settlage, J. Shabanowitz, S. A. Rosenberg, D. F. Hunt, and S. L. Topalian. 1999. Biochemical identification of a mutated human melanoma antigen recognized by CD4(+) T cells. J. Exp. Med. 189:757-766.

Robbins, P. F., M. El-Gamil, Y. F. Li, Y. Kawakami, D. Loftus, E. Appella, and S. A. Rosenberg. 1996. A mutated beta-catenin gene encodes a melanoma-specific antigen recognized by tumor infiltrating lymphocytes. J. Exp. Med. 183:1185-1192.

Roman, J., R. M. LaChance, T. J. Broekelmann, C. J. Kennedy, E. A. Wayner, W. G. Carter, and J. A. McDonald. 1989. The fibronectin receptor is organized by extracellular matrix fibronectin: implications for oncogenic transformation and for cell recognition of fibronectin matrices. J. Cell. Biol. 108:2529-2543.

Takahashi, T., P. B. Chapman, S. Y. Yang, I. Hara, S. Vijayasaradhi, and A. N. Houghton. 1995. Reactivity of autologous CD4+ T lymphocytes against human melanoma. Evidence for a shared melanoma antigen presented by HLA-DR15. J. Immunol. 154:772-779.

Toes, R. E., F. Ossendorp, R. Offringa, and C. J. Melief. 1999. CD4 T Cells and their role in antitumor inmmune responses. J. Exp. Med. 189:753-756.

Topalian, S. L., M. I. Gonzales, M. Parkhurst, Y. F. Li, S. Southwood, A. Sette, S. A. Rosenberg, and P. F. Robbins. 1996. Melanoma-specific CD4+ T cells recognize nonmutated HLA-DR-restricted tyrosinase epitopes. J. Exp. Med. 183:1965-1971.

Touloukian, C. E., W. W. Leitner, S. L. Topalian, Y. F. Li, P. F. Robbins, S. A. Rosenberg, and N. P. Restifo. 2000. Identification of a MHC class II-restricted human gp100 epitope using DR4-IE transgenic mice. J Immunol 164:3535-3542.

Van de Keere, F., and S. Tonegawa. 1998. CD4(+) T cells prevent spontaneous experimental autoimmune encephalomyelitis in anti-myelin basic protein T cell receptor transgenic mice. J. Exp. Med. 188:1875-1882.

Wang, R.-F. 2001. The role of MHC class II-restricted tumor antigens and CD4+ T cells in antitumor immunity. Trends in Immunology 22:269-276.

Wang, R.-F., and S. A. Rosenberg. 1999. Human tumor antigens for cancer vaccine development. Immunol. Rev. 170:85-100.

Wang, R.-F., E. Appella, Y. Kawakami, X. Kang, and S. A. Rosenberg. 1996. Identification of TRP-2 as a human tumor antigen recognized by cytotoxic T lymphocytes. J. Exp. Med. 184:2207-2216.

Wang, R.-F., S. L. Johnston, G. Zeng, D. J. Schwartzentruber, and S. A. Rosenberg. 1998. A breast and melanoma-shared tumor antigen: T cell responses to antigenic peptides translated from different open reading frames. J. Immunol. 161:3596-3606.

Wang, R.-F., X. Wang, A. C. Atwood, S. L. Topalian, and S. A. Rosenberg. 1999. Cloning genes encoding MHC class II-restricted antigens: mutated CDC27 as a tumor antigen. Science 284:1351-1354.

Wang, R.-F., X. Wang, and S. A. Rosenberg. 1999. Identification of a novel MHC class II-restricted tumor antigen resulting from a chromosomal rearrangement recognized by CD4+ T cells. J. Exp. Med. 189:1659-1667.

Wolfel, T., M. Hauer, J. Schneider, M. Serrano, C. Wolfel, E. Klehmann-Hieb, E. De Plaen, T. Hankeln, K.-H. Meyer Zum Buschenfelde, and D. Beach. 1995. A p16INK4a-insensitive CDK4 mutant targeted by cytolytic T lymphocytes in a human melanoma. Science 269:1281-1284.

Zarour, H. M., J. M. Kirkwood, L. S. Kierstead, W. Herr, V. Brusic, C. L. Slingluff, Jr., J. Sidney, A. Sette, and W. J. Storkus. 2000. Melan-AJMART-1(51-73) represents an immunogenic HLA-DR4-restricted epitope recognized by melanoma-reactive CD4(+) T cells. Proc. Natl. Acad. Sci. U.S.A. 97:400-405.

Zeng, G., C. E. Touloukian, X. Wang, N. P. Restifo, S. A. Rosenberg, and R.-F. Wang. 2000. Identification of CD4+ T cell epitopes from NY-ESO-1 presented by HLA-DR molecules. J. Immunol. 165:1153-1159.

Zeng, G., X. Wang, P. F. Robbins, S. A. Rosenberg, and R.-F. Wang. 2001. CD4+ T cell recognition of MHC class II-restricted epitopes from NY-ESO-1 presented by a prevalent HLA-DP4 allele: association with NY-ESO-1 antibody production. Proc. Natl. Acad. Sci. U.S.A. 98:3964-3969.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of identifying a cell that will differentiate into a metastatic cancer cell, comprising the step of identifying a mutated fibronectin in said cell.

2. The method of claim 1, wherein said cell is in a tumor.

3. The method of claim 1, wherein said mutated fibronectin is a mutated fibronectin polynucleotide.

4. The method of claim 3, wherein said mutated fibronectin polynucleotide comprises a G to A mutation at position 6427.

5. The method of claim 1, wherein said mutated fibronectin is a mutated fibronectin polypeptide.

6. The method of claim 5, wherein said mutated fibronectin polypeptide comprises a Glu to Lys substitution at position 2053.

7. The method of claim 5, wherein said mutated fibronectin polypeptide comprises the sequence MIFEKHGFRRTTPP (SEQ ID NO:1).

8. The method of claim 1, wherein said metastatic tumor cell is a melanoma cell, a prostate cancer cell, a breast cancer cell, a lung cancer cell, an ovarian cancer cell, a brain cancer cell, a liver cancer cell, a colon cancer cell, or a kidney cancer cell.

9. The method of claim 8, wherein said metastatic tumor cell is a melanoma cell.

10. A method of predicting metastasis from a cancer, comprising the step of identifying a mutated fibronectin polynucleotide or polypeptide in at least one cell of the cancer.

11. The method of claim 10, wherein the mutated fibronectin polynucleotide comprises a G to A mutation at position 6427.

12. The method of claim 10, wherein the mutated fibronectin polypeptide comprises a Glu to Lys substitution at position 2053.

13. The method of claim 10, wherein the mutated fibronectin polypeptide comprises the sequence MIFEKHGFRRTTPP (SEQ ID NO:1).

14. The method of claim 10, wherein the cancer is melanoma.

15. A method of eliciting an immune response in an individual, comprising the step of delivering to the individual a vector comprising a mutant fibronectin.

16. The method of claim 15, wherein the mutant fibronectin is further defined as a mutant fibronectin polynucleotide.

17. The method of claim 16, wherein the mutant fibronectin polynucleotide comprises a G to A mutation at position 6427.

18. The method of claim 15, wherein the mutant fibronectin is further defined as a mutant fibronectin polypeptide.

19. The method of claim 18, wherein the mutant fibronectin polypeptide comprises a Glu to Lys substitution at position 2053.

20. The method of claim 18, wherein the mutant fibronectin polypeptide is MIFEKHGFRRTTPP (SEQ ID NO:1).

21. The method of claim 15, wherein the mutant fibronectin is delivered to the individual in a cell.

22. The method of claim 21, wherein the cell is an immune effector cell.

23. The method of claim 22, wherein the immune effector cell is a dendritic cell.

24. The method of claim 22, wherein following the delivery of the mutant fibronectin to the immune effector cell, at least part of a mutant fibronectin polypeptide is presented on the cell surface.

25. The method of claim 15, wherein the method further comprises the step of delivering a tumor antigen other than mutant fibronectin to the individual.

26. The method of claim 25, wherein the tumor antigen other than mutant fibronectin is delivered to the individual in a cell.

27. The method of claim 25, wherein the tumor antigen is TRP-2 or NY-ESO-1.

28. A method of preventing metastasis of a cancer in an individual, wherein the individual comprises at least one cell having a mutated fibronectin, comprising the step of stimulating an immune response against at least said cell.

29. The method of claim 28, wherein said immune response is further defined as being against said mutated fibronectin.

30. The method of claim 28, wherein the cell having a mutated fibronectin is a cancer cell.

31. The method of claim 28, wherein said stimulating an immune response is further defined as comprising the steps of:
introducing the mutated fibronectin to a dendritic cell; and
administering the cell comprising the mutated fibronectin to the individual, wherein the dendritic cell presents at least part of the mutated fibronectin on its surface.

32. An isolated mutant fibronectin polynucleotide comprising a G to A mutation at position 6427.

33. An isolated mutant fibronectin polypeptide comprising a Glu to Lys substitution at position 2053.

34. An isolated mutant fibronectin polypeptide comprising MIFEKHGFRRTTPP (SEQ ID NO:1).

35. A pharmaceutical composition comprising a mutant fibronectin polynucleotide having a G to A mutation at position 6427.

36. The composition of claim 35, wherein the polynucleotide is in a pharmaceutically acceptable carrier.

37. A pharmaceutical composition comprising a mutant fibronectin polypeptide having a Glu to Lys substitution at position 2053.

38. The composition of claim 37, wherein said polypeptide comprises MIFEKHGFRRTTPP (SEQ ID NO:1).

39. The composition of claim 37, wherein the polypeptide is in a pharmaceutically acceptable carrier.

40. An immunological composition comprising a mutant fibronectin polynucleotide having a G to A mutation at position 6427.

41. An immunological composition comprising a mutant fibronectin polypeptide having a Glu to Lys substitution at position 2053.

42. The composition of claim 41, wherein said polypeptide comprises MIFEKHGFRRTTPP (SEQ ID NO: 1).

43. An immune effector cell comprising a mutant fibronectin polynucleotide having a G to A mutation at position 6427.

44. The cell of claim 43, wherein said cell is a dendritic cell.

45. An immune effector cell comprising a mutant fibronectin polypeptide having a Glu to Lys substitution at position 2053.

46. The cell of claim 45, wherein said polypeptide comprises MIFEKHGFRRTTPP (SEQ ID NO:1).

47. The cell of claim 45, wherein said cell is a dendritic cell.

48. An antigen presenting cell transduced with a vector comprising a mutant fibronectin polynucleotide having a G to A mutation at position 6427.

49. An antigen presenting cell comprising a fibronectin polypeptide having the sequence MIFEKHGFRRTTPP (SEQ ID NO: 1).


SEQUENCE LISTING (148 sequences)

SEQ ID NO: 1 - protein, 14 aa, Human
MIFEKHGFRRTTPP

SEQ ID NO: 2 - DNA, 2384 bp, Human
atcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggtgttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggtgaggaaatccaaattggtcacattcccagggaagatgtagactatcacctgtacccacacggtccggggctcaatccaaatgcctctacaggacaagaagctctctctcagacaaccatctcatgggccccattccaggacacttctgagtacatcatttcatgtcatcctgttggcactgatgaagaacccttacagttcagggttcctggaacttctaccagtgcgactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttacgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaa

SEQ ID NO: 3 - protein, 2355 aa, Human
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDPTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGQEALSQTTISWAPFQDTSEYIISCHPVGTDEEPLQFRVPGTSTSATLTGLTRGATYNIIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 4 - DNA, 7705 bp, Human
gaagagcaagaggcaggctcagcaaatggttcagccccagtccccggtggctgtcagtcaaagcaagcccggttgttatgacaatggaaaacactatcagataaatcaacagtgggagcggacctacctaggtaatgtgttggtttgtacttgttatggaggaagccgaggttttaactgcgaaagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggactccctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaatttgagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggttgaagacacaaggaaataagcaaatgctttgcacgtgcctgggcaacggagtcagctgccaagagacagctgtaacccagacttacggtggcaacttaaatggagagccatgtgtcttaccattcacctacaatggcaggacgttctactcctgcaccacggaagggcgacaggacggacatctttggtgcagcacaacttcgaattatgagcaggaccagaaatactctttctgcacagaccacactgttttggttcagactcaaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcaacttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggccagcccaggcagtacaatgtgggtccctctgtcttcaagtaccccctgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccatcgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggtgttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttacgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccacaaaaaaaaaaaaaaaaaaaaaaaaaa

SEQ ID NO: 5 - DNA, 33 bp, Artificial Sequence
  Synthetic Primer
ctcaacatggttaggggtccggggcccgggctg

SEQ ID NO: 6 - DNA, 23 bp, Artificial Sequence
  Synthetic Primer
agagacatgcttgttcctctgga

SEQ ID NO: 7 - protein, 9 aa, Artificial Sequence
  Synthetic Peptide
SVYDFFVWL

SEQ ID NO: 8 - DNA, 8027 bp, Human
acgcccgcgccggctgtgctgcacagggggaggagagggaaccccaggcgcgagcgggaagaggggacctgcagccacaacttctctggtcctctgcatcccttctgtccctccacccgtccccttccccaccctctggcccccaccttcttggaggcgacaacccccgggaggcattagaagggatttttcccgcagttgcgaagggaagcaaacttggtggcaacttgcctcccggtgcgggcgtctctcccccaccgtctcaacatgcttaggggtccggggcccgggctgctgctgctggccgtccagtgcctggggacagcggtgccctccacgggagcctcgaagagcaagaggcaggctcagcaaatggttcagccccagtccccggtggctgtcagtcaaagcaagcccggttgttatgacaatggaaaacactatcagataaatcaacagtgggagcggacctacctaggcaatgcgttggtttgtacttgttatggaggaagccgaggttttaactgcgagagtaaacctgaagctgaagagacttgctttgacaagtacactgggaacacttaccgagtgggtgacacttatgagcgtcctaaagactccatgatctgggactgtacctgcatcggggctgggcgagggagaataagctgtaccatcgcaaaccgctgccatgaagggggtcagtcctacaagattggtgacacctggaggagaccacatgagactggtggttacatgttagagtgtgtgtgtcttggtaatggaaaaggagaatggacctgcaagcccatagctgagaagtgttttgatcatgctgctgggacttcctatgtggtcggagaaacgtgggagaagccctaccaaggctggatgatggtagattgtacttgcctgggagaaggcagcggacgcatcacttgcacttctagaaatagatgcaacgatcaggacacaaggacatcctatagaattggagacacctggagcaagaaggataatcgaggaaacctgctccagtgcatctgcacaggcaacggccgaggagagtggaagtgtgagaggcacacctctgtgcagaccacatcgagcggatctggccccttcaccgatgttcgtgcagctgtttaccaaccgcagcctcacccccagcctcctccctatggccactgtgtcacagacagtggtgtggtctactctgtggggatgcagtggctgaagacacaaggaaataagcaaatgctttgcacgtgcctgggcaacggagtcagctgccaagagacagctgtaacccagacttacggtggcaactcaaatggagagccatgtgtcttaccattcacctacaatggcaggacgttctactcctgcaccacggaagggcgacaggacggacatctttggtgcagcacaacttcgaattatgagcaggaccagaaatactctttctgcacagaccacactgttttggttcagactcgaggaggaaattccaatggtgccttgtgccacttccccttcctatacaacaaccacaattacactgattgcacttctgagggcagaagagacaacatgaagtggtgtgggaccacacagaactatgatgccgaccagaagtttgggttctgccccatggctgcccacgaggaaatctgcacaaccaatgaaggggtcatgtaccgcattggagatcagtgggataagcagcatgacatgggtcacatgatgaggtgcacgtgtgttgggaatggtcgtggggaatggacatgcattgcctactcgcagcttcgagatcagtgcattgttgatgacatcacttacaatgtgaacgacacattccacaagcgtcatgaagaggggcacatgctgaactgtacatgcttcggtcagggtcggggcaggtggaagtgtgatcccgtcgaccaatgccaggattcagagactgggacgttttatcaaattggagattcatgggagaagtatgtgcatggtgtcagataccagtgctactgctatggccgtggcattggggagtggcattgccaacctttacagacctatccaagctcaagtggtcctgtcgaagtatttatcactgagactccgagtcagcccaactcccaccccatccagtggaatgcaccacagccatctcacatttccaagtacattctcaggtggagacctaaaaattctgtaggccgttggaaggaagctaccataccaggccacttaaactcctacaccatcaaaggcctgaagcctggtgtggtatacgagggccagctcatcagcatccagcagtacggccaccaagaagtgactcgctttgacttcaccaccaccagcaccagcacacctgtgaccagcaacaccgtgacaggagagacgactcccttttctcctcttgtggccacttctgaatctgtgaccgaaatcacagccagtagctttgtggtctcctgggtctcagcttccgacaccgtgtcgggattccgggtggaatatgagctgagtgaggagggagatgagccacagtacctggatcttccaagcacagccacttctgtgaacatccctgacctgcttcctggccgaaaatacattgtaaatgtctatcagatatctgaggatggggagcagagtttgatcctgtctacttcacaaacaacagcgcctgatgcccctcctgacccgactgtggaccaagttgatgacacctcaattgttgttcgctggagcagaccccaggctcccatcacagggtacagaatagtctattcgccatcagtagaaggtagcagcacagaactcaaccttcctgaaactgcaaactccgtcaccctcagtgacttgcaacctggtgttcagtataacatcactatctatgctgtggaagaaaatcaagaaagtacacctgttgtcattcaacaagaaaccactggcaccccacgctcagatacagtgccctctcccagggacctgcagtttgtggaagtgacagacgtgaaggtcaccatcatgtggacaccgcctgagagtgcagtgaccggctaccgtgtggatgtgatccccgtcaacctgcctggcgagcacgggcagaggctgcccatcagcaggaacacctttgcagaagtcaccgggctgtcccctggggtcacctattacttcaaagtctttgcagtgagccatgggagggagagcaagcctctgactgctcaacagacaaccaaactggatgctcccactaacctccagtttgtcaatgaaactgattctactgtcctggtgagatggactccacctcgggcccagataacaggataccgactgaccgtgggccttacccgaagaggccagcccaggcagtacaatgtgggtccctctgtctccaagtaccccctgaggaatctgcagcctgcatctgagtacaccgtatccctcgtggccataaagggcaaccaagagagccccaaagccactggagtctttaccacactgcagcctgggagctctattccaccttacaacaccgaggtgactgagaccaccatcgtgatcacatggacgcctgctccaagaattggttttaagctgggtgtacgaccaagccagggaggagaggcaccacgagaagtgacttcagactcaggaagcatcgttgtgtccggcttgactccaggagtagaatacgtctacaccatccaagtcctgagagatggacaggaaagagatgcgccaattgtaaacaaagtggtgacaccattgtctccaccaacaaacttgcatctggaggcaaaccctgacactggagtgctcacagtctcctgggagaggagcaccaccccagacattactggttatagaattaccacaacccctacaaacggccagcagggaaattctttggaagaagtggtccatgctgatcagagctcctgcacttttgataacctgagtcccggcctggagtacaatgtcagtgtttacactgtcaaggatgacaaggaaagtgtccctatctctgataccatcatcccagctgttcctcctcccactgacctgcgattcaccaacattggtccagacaccatgcgtgtcacctgggctccacccccatccattgatttaaccaacttcctggtgcgttactcacctgtgaaaaatgaggaagatgttgcagagttgtcaatttctccttcagacaatgcagtggtcttaacaaatctcctgcctggtacagaatatgtagtgagtgtctccagtgtctacgaacaacatgagagcacacctcttagaggaagacagaaaacaggtcttgattccccaactggcattgacttttctgatattactgccaactcttttactgtgcactggattgctcctcgagccaccatcactggctacaggatccgccatcatcccgagcacttcagtgggagacctcgagaagatcgggtgccccactctcggaattccatcaccctcaccaacctcactccaggcacagagtatgtggtcagcatcgttgctcttaatggcagagaggaaagtcccttattgattggccaacaatcaacagtttctgatgttccgagggacctggaagttgttgctgcgacccccaccagcctactgatcagctgggatgctcctgctgtcacagtgagatattacaggatcacttacggagaaacaggaggaaatagccctgtccaggagttcactgtgcctgggagcaagtctacagctaccatcagcggccttaaacctggagttgattataccatcactgtgtatgctgtcactggccgtggagacagccccgcaagcagcaagccaatttccattaattaccgaacagaaattgacaaaccatcccagatgcaagtgaccgatgttcaggacaacagcattagtgtcaagtggctgccttcaagttcccctgttactggttacagagtaaccaccactcccaaaaatggaccaggaccaacaaaaactaaaactgcaggtccagatcaaacagaaatgactattgaaggcttgcagcccacagtggagtatgtggttagtgtctatgctcagaatccaagcggagagagtcagcctctggttcagactgcagtaaccaacattgatcgccctaaaggactggcattcactgatgtggatgtcgattccatcaaaattgcttgggaaagcccacaggggcaagtttccaggtacagggtgacctactcgagccctgaggatggaatccatgagctattccctgcacctgatggtgaagaagacactgcagagctgcaaggcctcagaccgggttctgagtacacagtcagtgtggttgccttgcacgatgatatggagagccagcccctgattggaacccagtccacagctattcctgcaccaactgacctgaagttcactcaggtcacacccacaagcctgagcgcccagtggacaccacccaatgttcagctcactggatatcgagtgcgggtgacccccaaggagaagaccggaccaatgaaagaaatcaaccttgctcctgacagctcatccgtggttgtatcaggacttatggtggccaccaaatatgaagtgagtgtctatgctcttaaggacactttgacaagcagaccagctcagggtgttgtcaccactctggagaatgtcagcccaccaagaagggctcgtgtgacagatgctactgagaccaccatcaccattagctggagaaccaagactgagacgatcactggcttccaagttgatgccgttccagccaatggccagactccaatccagagaaccatcaagccagatgtcagaagctacaccatcacaggtttacaaccaggcactgactacaagatctacctgtacaccttgaatgacaatgctcggagctcccctgtggtcatcgacgcctccactgccattgatgcaccatccaacctgcgtttcctggccaccacacccaattccttgctggtatcatggcagccgccacgtgccaggattaccggctacatcatcaagtatgagaagcctgggtctcctcccagagaagtggtccctcggccccgccctggtgtcacagaggctactattactggcctggaaccgggaaccgaatatacaatttatgtcattgccctgaagaataatcagaagagcgagcccctgattggaaggaaaaagacagacgagcttccccaactggtaacccttccacaccccaatcttcatggaccagagatcttggatgttccttccacagttcaaaagacccctttcgtcacccaccctgggtatgacactggaaatggtattcagcttcctggcacttctggtcagcaacccagtgttgggcaacaaatgatctttgaggaacatggttttaggcggaccacaccgcccacaacggccacccccataaggcataggccaagaccatacccgccgaatgtaggacaagaagctctctctcagacaaccatctcatgggccccattccaggacacttctgagtacatcatttcatgtcatcctgttggcactgatgaagaacccttacagttcagggttcctggaacttctaccagtgccactctgacaggcctcaccagaggtgccacctacaacatcatagtggaggcactgaaagaccagcagaggcataaggttcgggaagaggttgttaccgtgggcaactctgtcaacgaaggcttgaaccaacctacggatgactcgtgctttgacccctacacagtttcccattatgccgttggagatgagtgggaacgaatgtctgaatcaggctttaaactgttgtgccagtgcttaggctttggaagtggtcatttcagatgtgattcatctagatggtgccatgacaatggtgtgaactacaagattggagagaagtgggaccgtcagggagaaaatggccagatgatgagctgcacatgtcttgggaacggaaaaggagaattcaagtgtgaccctcatgaggcaacgtgttacgatgatgggaagacataccacgtaggagaacagtggcagaaggaatatctcggtgccatttgctcctgcacatgctttggaggccagcggggctggcgctgtgacaactgccgcagacctgggggtgaacccagtcccgaaggcactactggccagtcctacaaccagtattctcagagataccatcagagaacaaacactaatgttaattgcccaattgagtgcttcatgcctttagatgtacaggctgacagagaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctaagatttggtttgggatcaataggaaagcatatgcagccaaccaagatgcaaatgttttgaaatgatatgaccaaaattttaagtaggaaagtcacccaaacacttctgctttcacttaagtgtctggcccgcaatactgtaggaacaagcatgatcttgttactgtgatattttaaatatccacagtactcactttttccaaatgatcctagtaattgcctagaaatatctttctcttacctgttatttatcaatttttcccagtatttttatacggaaaaaattgtattgaaaacacttagtatgcagttgataagaggaatttggtataattatggtgggtgattattttttatactgtatgtgccaaagctttactactgtggaaagacaactgttttaataaaagatttacattccaca

SEQ ID NO: 9 - protein, 9 aa, Artificial Sequence
  Synthetic Peptide
EADPTGHSY

SEQ ID NO: 10 - protein, 9 aa, Artificial Sequence
  Synthetic Peptide
SLFRAVITK

SEQ ID NO: 11 - protein, 9 aa, Artificial Sequence
  Synthetic Peptide
NYKHCFPEI

SEQ ID NO: 12 - protein, 10 aa, Artificial Sequence
  Synthetic Peptide
EVYDGREHSA

SEQ ID NO: 13 - protein, 10 aa, Artificial Sequence
  Synthetic Peptide
REPVTKAEML

SEQ ID NO: 14 - protein, 9 aa, Artificial Sequence
  Synthetic Peptide
DPARYEFLW

SEQ ID NO: 15 - protein, 9 aa, Artificial Sequence
  Synthetic Peptide
SAFPTTINF

SEQ ID NO: 16 - protein, 9 aa, Artificial Sequence
  Synthetic Peptide
SAYGEPRKL

SEQ ID NO: 17 - protein, 9 aa, Artificial Sequence
  Synthetic Peptide
SAYGEPRKL

SEQ ID NO: 18 - protein, 9 aa, Artificial Sequence
  Synthetic Peptide
KMVELVHFL

SEQ ID NO: 19 - protein, 9 aa, Artificial Sequence
  Synthetic Peptide
YLQVFGIEV

SEQ ID NO: 20 - protein, 9 aa, Artificial Sequence
  Synthetic Peptide
EYLQLVFGI

SEQ ID NO: 21 - protein, 10 aa, Artificial Sequence
  Synthetic Peptide
EADIPIGHLY

SEQ ID NO: 22 - protein, 9 aa, Artificial Sequence
  Synthetic Peptide
FLWGPRALV

SEQ ID NO: 23 - protein, 9 aa, Artificial Sequence
  Synthetic Peptide
TFPDLESEF

SEQ ID NO: 24 - protein, 9 aa, Artificial Sequence
  Synthetic Peptide
IMPKAGLLI

SEQ ID NO: 25 - protein, 10 aa, Artificial Sequence
  Synthetic Peptide
MEVDPIGHLY

SEQ ID NO: 26 - protein, 9 aa, Artificial Sequence
  Synthetic Peptide
WQYFFPVIF

SEQ ID NO: 27 - protein, 10 aa, Artificial Sequence
  Synthetic Peptide
GVYDGREHTV

SEQ ID NO: 28 - protein, 9 aa, Artificial Sequence
  Synthetic Peptide
MVKISGGPR

SEQ ID NO: 29 - protein, 9 aa, Artificial Sequence
  Synthetic Peptide
GLYDGMEHL

SEQ ID NO: 30 - protein, 9 aa, Artificial Sequence
  Synthetic Peptide
VRIGHLYIL

SEQ ID NO: 31 - protein, 9 aa, Artificial Sequence
  Synthetic Peptide
AARAVFLAL

SEQ ID NO: 32 - protein, 9 aa, Artificial Sequence
  Synthetic Peptide
FLWGPRAYA

SEQ ID NO: 33 - protein, 8 aa, Artificial Sequence
  Synthetic Peptide
YRPRPRRY

SEQ ID NO: 34 - protein, 9 aa, Artificial Sequence
  Synthetic Peptide
YYWPRPRRY

SEQ ID NO: 35 - protein, 11 aa, Artificial Sequence
  Synthetic Peptide
MTQGQHFLQKV

SEQ ID NO: 36 - protein, 11 aa, Artificial Sequence
  Synthetic Peptide
SLLMWITQCFL

SEQ ID NO: 37 - protein, 9 aa, Artificial Sequence
  Synthetic Peptide
SLLMWITQC

SEQ ID NO: 38 - protein, 9 aa, Artificial Sequence
  Synthetic Peptide
QLSLLMWIT

SEQ ID NO: 39 - protein, 10 aa, Artificial Sequence
  Synthetic Peptide
ASGPGGGAPR

SEQ ID NO: 40 - protein, 9 aa, Artificial Sequence
  Synthetic Peptide
AAGIGILTV

SEQ ID NO: 41 - protein, 10 aa, Artificial Sequence
  Synthetic Peptide
EAAGIGILTV

SEQ ID NO: 42 - protein, 9 aa, Artificial Sequence
  Synthetic Peptide
ILTVILGVL

SEQ ID NO: 43 - protein, 10 aa, Artificial Sequence
  Synthetic Peptide
AEEAAGIGIL

SEQ ID NO: 44 - protein, 11 aa, Artificial Sequence
  Synthetic Peptide
AEEAAGIGILT

SEQ ID NO: 45 - protein, 9 aa, Artificial Sequence
  Synthetic Peptide
TILLGIFFL

SEQ ID NO: 46 - protein, 9 aa, Artificial Sequence
  Synthetic Peptide
FLALIICNA

SEQ ID NO: 47 - protein, 9 aa, Artificial Sequence
  Synthetic Peptide
KTWGQYWQV

SEQ ID NO: 48 - protein, 10 aa, Artificial Sequence
  Synthetic Peptide
AMLGTHTMEV

SEQ ID NO: 49 - protein, 9 aa, Artificial Sequence
  Synthetic Peptide
MLGTHTMEV

SEQ ID NO: 50 - protein, 10 aa, Artificial Sequence
  Synthetic Peptide
SLADTNSLAV

SEQ ID NO: 51 - protein, 9 aa, Artificial Sequence
  Synthetic Peptide
ITDQVPFSV

SEQ ID NO: 52 - protein, 10 aa, Artificial Sequence
  Synthetic Peptide
LLDGTATLRL

SEQ ID NO: 53 - protein, 9 aa, Artificial Sequence
  Synthetic Peptide
YLEPGPVTA

SEQ ID NO: 54 - protein, 10 aa, Artificial Sequence
  Synthetic Peptide
VLYRYGSFSV

SEQ ID NO: 55 - protein, 9 aa, Artificial Sequence
  Synthetic Peptide
RLMKQDFSV

SEQ ID NO: 56 - protein, 9 aa, Artificial Sequence
  Synthetic Peptide
FLPRIFCSC

SEQ ID NO: 57 - protein, 9 aa, Artificial Sequence
  Synthetic Peptide
LIYRRRLMK

SEQ ID NO: 58 - protein, 9 aa, Artificial Sequence
  Synthetic Peptide
ALNFPGSQK

SEQ ID NO: 59 - protein, 10 aa, Artificial Sequence
  Synthetic Peptide
SLIYRRRLMK

SEQ ID NO: 60 - protein, 9 aa, Artificial Sequence
  Synthetic Peptide
ALLAVGATK

SEQ ID NO: 61 - protein, 9 aa, Artificial Sequence
  Synthetic Peptide
VYFFLPDHL

SEQ ID NO: 62 - protein, 8 aa, Artificial Sequence
  Synthetic Peptide
SNDGPTLI

SEQ ID NO: 63 - protein, 10 aa, Artificial Sequence
  Synthetic Peptide
VSHSFPHPLY

SEQ ID NO: 64 - protein, 10 aa, Artificial Sequence
  Synthetic Peptide
FLTPKKLQCV

SEQ ID NO: 65 - protein, 10 aa, Artificial Sequence
  Synthetic Peptide
VISNDVCAQV

SEQ ID NO: 66 - protein, 10 aa, Artificial Sequence
  Synthetic Peptide
HSTNGVTRIY

SEQ ID NO: 67 - protein, 9 aa, Artificial Sequence
  Synthetic Peptide
KCDICTDEY

SEQ ID NO: 68 - protein, 10 aa, Artificial Sequence
  Synthetic Peptide
SSYVIPIGTY

SEQ ID NO: 69 - protein, 9 aa, Artificial Sequence
  Synthetic Peptide
YMDGTMSQV

SEQ ID NO: 70 - protein, 9 aa, Artificial Sequence
  Synthetic Peptide
MLLAVLYCL

SEQ ID NO: 71 - protein, 9 aa, Artificial Sequence
  Synthetic Peptide
AFLPWHRLF

SEQ ID NO: 72 - protein, 9 aa, Artificial Sequence
  Synthetic Peptide
SETWRDIDF

SEQ ID NO: 73 - protein, 9 aa, Artificial Sequence
  Synthetic Peptide
MSLQRQFLR

SEQ ID NO: 74 - protein, 9 aa, Artificial Sequence
  Synthetic Peptide
SVYDFFVWL

SEQ ID NO: 75 - protein, 9 aa, Artificial Sequence
  Synthetic Peptide
TLDSQVMSL

SEQ ID NO: 76 - protein, 9 aa, Artificial Sequence
  Synthetic Peptide
LLGPGRPYR

SEQ ID NO: 77 - protein, 9 aa, Artificial Sequence
  Synthetic Peptide
LLGPGRPYR

SEQ ID NO: 78 - protein, 9 aa, Artificial Sequence
  Synthetic Peptide
ANDPIFVVL

SEQ ID NO: 79 - protein, 8 aa, Artificial Sequence
  Synthetic Peptide
AFLRHAAL

SEQ ID NO: 80 - protein, 10 aa, Artificial Sequence
  Synthetic Peptide
DYPSLSATDI

SEQ ID NO: 81 - protein, 11 aa, Artificial Sequence
  Synthetic Peptide
MLMAQEALAFL

SEQ ID NO: 82 - protein, 11 aa, Artificial Sequence
  Synthetic Peptide
MLMAQEALAFL

SEQ ID NO: 83 - protein, 9 aa, Artificial Sequence
  Synthetic Peptide
HLFGYSWYK

SEQ ID NO: 84 - protein, 9 aa, Artificial Sequence
  Synthetic Peptide
KFHRVIKDF

SEQ ID NO: 85 - protein, 9 aa, Artificial Sequence
  Synthetic Peptide
DFMIQGGDF

SEQ ID NO: 86 - protein, 9 aa, Artificial Sequence
  Synthetic Peptide
KIFGSLAFL

SEQ ID NO: 87 - protein, 9 aa, Artificial Sequence
  Synthetic Peptide
IISAVVGIL

SEQ ID NO: 88 - protein, 9 aa, Artificial Sequence
  Synthetic Peptide
RLLQETELV

SEQ ID NO: 89 - protein, 9 aa, Artificial Sequence
  Synthetic Peptide
VVLGVVFGI

SEQ ID NO: 90 - protein, 9 aa, Artificial Sequence
  Synthetic Peptide
ILHNGAYSL

SEQ ID NO: 91 - protein, 10 aa, Artificial Sequence
  Synthetic Peptide
YMIMVKCWMI

SEQ ID NO: 92 - protein, 9 aa, Artificial Sequence
  Synthetic Peptide
VLRENTSPK

SEQ ID NO: 93 - protein, 9 aa, Artificial Sequence
  Synthetic Peptide
ILAKFLHWL

SEQ ID NO: 94 - protein, 9 aa, Artificial Sequence
  Synthetic Peptide
ILAKFLHWL

SEQ ID NO: 95 - protein, 9 aa, Artificial Sequence
  Synthetic Peptide
RLVDDFLLV

SEQ ID NO: 96 - protein, 9 aa, Artificial Sequence
  Synthetic Peptide
SPRWWPTCL

SEQ ID NO: 97 - protein, 9 aa, Artificial Sequence
  Synthetic Peptide
STAPPAHGV

SEQ ID NO: 98 - protein, 9 aa, Artificial Sequence
  Synthetic Peptide
STAPPVHNV

SEQ ID NO: 99 - protein, 9 aa, Artificial Sequence
  Synthetic Peptide
LLNQLQVNL

SEQ ID NO: 100 - protein, 9 aa, Artificial Sequence
  Synthetic Peptide
MLWGWREHV

SEQ ID NO: 101 - protein, 9 aa, Artificial Sequence
  Synthetic Peptide
LYVDSLFFL

SEQ ID NO: 102 - protein, 9 aa, Artificial Sequence
  Synthetic Peptide
AYGLDFYIL

SEQ ID NO: 103 - protein, 9 aa, Artificial Sequence
  Synthetic Peptide
VPYGSFKHV

SEQ ID NO: 104 - protein, 9 aa, Artificial Sequence
  Synthetic Peptide
LPRWPPPQL

SEQ ID NO: 105 - protein, 9 aa, Artificial Sequence
  Synthetic Peptide
EYRGFTQDF

SEQ ID NO: 106 - protein, 9 aa, Artificial Sequence
  Synthetic Peptide
KGSGKMKTE

SEQ ID NO: 107 - protein, 10 aa, Artificial Sequence
  Synthetic Peptide
VYDYNCHVDL

SEQ ID NO: 108 - protein, 9 aa, Artificial Sequence
  Synthetic Peptide
AYIDFEMKI

SEQ ID NO: 109 - protein, 9 aa, Artificial Sequence
  Synthetic Peptide
RMFPNAPYL

SEQ ID NO: 110 - protein, 9 aa, Artificial Sequence
  Synthetic Peptide
GVALQTMKQ

SEQ ID NO: 111 - protein, 9 aa, Artificial Sequence
  Synthetic Peptide
SYLDSGIHF

SEQ ID NO: 112 - protein, 9 aa, Artificial Sequence
  Synthetic Peptide
FPSDSWCYF

SEQ ID NO: 113 - protein, 10 aa, Artificial Sequence
  Synthetic Peptide
ACDPHSGHFV

SEQ ID NO: 114 - protein, 9 aa, Artificial Sequence
  Synthetic Peptide
ETVSEQSNV

SEQ ID NO: 115 - protein, 10 aa, Artificial Sequence
  Synthetic Peptide
VLPDVFIRCV

SEQ ID NO: 116 - protein, 9 aa, Artificial Sequence
  Synthetic Peptide
HLSTAFARV

SEQ ID NO: 117 - protein, 9 aa, Artificial Sequence
  Synthetic Peptide
SLFEGIDIY

SEQ ID NO: 118 - protein, 13 aa, Artificial Sequence
  Synthetic Peptide
CVEWLRIYLENGK

SEQ ID NO: 119 - protein, 10 aa, Artificial Sequence
  Synthetic Peptide
YSWMDISCWI

SEQ ID NO: 120 - protein, 9 aa, Artificial Sequence
  Synthetic Peptide
AEPINIQTV

SEQ ID NO: 121 - protein, 9 aa, Artificial Sequence
  Synthetic Peptide
EEKLIVVLF

SEQ ID NO: 122 - protein, 10 aa, Artificial Sequence
  Synthetic Peptide
SELFRSGLDY

SEQ ID NO: 123 - protein, 9 aa, Artificial Sequence
  Synthetic Peptide
FRSGLDSYV

SEQ ID NO: 124 - protein, 9 aa, Artificial Sequence
  Synthetic Peptide
EAFIQPITR

SEQ ID NO: 125 - protein, 9 aa, Artificial Sequence
  Synthetic Peptide
KINKNPKYK

SEQ ID NO: 126 - protein, 10 aa, Artificial Sequence
  Synthetic Peptide
SPSSNRIRNT

SEQ ID NO: 127 - protein, 9 aa, Artificial Sequence
  Synthetic Peptide
DYSARWNEI

SEQ ID NO: 128 - protein, 9 aa, Artificial Sequence
  Synthetic Peptide
AYDFLYNYL

SEQ ID NO: 129 - protein, 9 aa, Artificial Sequence
  Synthetic Peptide
SYTRLFLIL

SEQ ID NO: 130 - protein, 10 aa, Artificial Sequence
  Synthetic Peptide
EVISCKLIKR

SEQ ID NO: 131 - protein, 9 aa, Artificial Sequence
  Synthetic Peptide
RVAALARDA

SEQ ID NO: 132 - protein, 16 aa, Artificial Sequence
  Synthetic Peptide
DVPKWISIMTERSVPH

SEQ ID NO: 133 - protein, 16 aa, Artificial Sequence
  Synthetic Peptide
WNRQLYPEWTEAQRLD

SEQ ID NO: 134 - protein, 14 aa, Artificial Sequence
  Synthetic Peptide
LLKYRARIPVTKAE

SEQ ID NO: 135 - protein, 15 aa, Artificial Sequence
  Synthetic Peptide
TSYVKVLHHMVKISG

SEQ ID NO: 136 - protein, 14 aa, Artificial Sequence
  Synthetic Peptide
AELVHFLLLKYRAR

SEQ ID NO: 137 - protein, 23 aa, Artificial Sequence
  Synthetic Peptide
RNGYRALMDKSLHVGTQCALTRR

SEQ ID NO: 138 - protein, 12 aa, Artificial Sequence
  Synthetic Peptide
PGSTAPPAHGVT

SEQ ID NO: 139 - protein, 10 aa, Artificial Sequence
  Synthetic Peptide
VLLKEFTVSG

SEQ ID NO: 140 - protein, 54 aa, Artificial Sequence
  Synthetic Peptide
PLPVPGVLLKEFTVSNGIVLLKEFTVSGNILTIRLTAADHRQLQLSISSCLQQL

SEQ ID NO: 141 - protein, 86 aa, Artificial Sequence
  Synthetic Peptide
ILLGRMSLFMPEDTGSLFHPEDTGQVFQQVFQVSHSFPHPLYDNDLMLLRLSEPAELTKKLQCVQLHVISMGVLQGITSMGSEPCA

SEQ ID NO: 142 - protein, 43 aa, Artificial Sequence
  Synthetic Peptide
QNILLSNAPLGPQFPDYSYLQDSDPDSFQDSYLQDSDPDSFQD

SEQ ID NO: 143 - protein, 21 aa, Artificial Sequence
  Synthetic Peptide
RHRPLQEVYPEANAPIGHNRE

SEQ ID NO: 144 - protein, 27 aa, Artificial Sequence
  Synthetic Peptide
SYNTHETICPEPTIDEEIWRDIDFAHE

SEQ ID NO: 145 - protein, 13 aa, Artificial Sequence
  Synthetic Peptide
LFMDTLSFVCPLC

SEQ ID NO: 146 - protein, 13 aa, Artificial Sequence
  Synthetic Peptide
LFMDSLNFVCPWC

SEQ ID NO: 147 - protein, 12 aa, Artificial Sequence
  Synthetic Peptide
FSWAMDLDPKGA

SEQ ID NO: 148 - protein, 15 aa, Artificial Sequence
  Synthetic Peptide
GELIGILNAAKVPAD